(19) United States
(12) Patent Application Publication
(10) Pub. No.: US 2026/0345697 A1
(43) Pub. Date: Nov. 5, 2026

(54) METHOD FOR PROVIDING RELIEF FROM LACTOSE INDUCED ABDOMINAL DISCOMFORT

(71) Applicant: Novozymes A/S, Bagsvaerd (DK)

(72) Inventors: Karolina TYKWINSKA, Berlin (DE); Natalia BOLOTINA, Berlin (DE); Sameer KULKARNI, Lyngby (DK)

(73) Assignee: Novozymes A/S, Bagsvaerd (DK)

(21) Appl. No.: 18/992,494

(22) PCT Filed: Jul. 12, 2023
(86) PCT No.: PCT/EP2023/069267

§ 371 Date: Jan. 8, 2025

(30) Foreign Application Priority Data
Jul. 12, 2022 (EP) 22184242.0

(51) Int. Cl. A61K 35/747; A61K 35/00; A61K 38/47; C12N 1/205; C12R 1/225

(52) U.S. Cl. CPC A61K 35/747; A61K 38/47; C12N 1/205; C12Y 302/01108; A61K 2035/115; C12R 2001/225

(57) ABSTRACT

The present invention relates to Ligilactobacillus salivarius strains to provide relief from or prevent lactose induced abdominal discomfort; digest lactose or improve digestion of lactose; provide digestive comfort after digestion of lactose on consumption of products containing lactose; and/or provide long-lasting support and/or effect for lactose digestion. The present invention also relates to use of Ligilactobacillus salivarius strains or a composition comprising such to provide relief from or prevent lactose induced abdominal discomfort; digest lactose or improve digestion of lactose; provide digestive comfort after digestion of lactose on consumption of products containing lactose; and/or provide long-lasting support and/or effect for lactose digestion to people suffering from lactose intolerance.

### CROSS-REFERENCE TO RELATED APPLICATIONS

This application is a 35 U.S.C. 371 national application of PCT/EP2023/069267, filed Jul. 12, 2023, which claims priority, or the benefit 35 U.S.C. 119 of European application no. 22184242.0 filed Jul. 12, 2022. The contents of these applications are fully incorporated herein by reference.

### REFERENCE TO SEQUENCE LISTING

This application contains a Sequence Listing in computer readable form created on Jul. 1, 2026, named 16010-WO-PCT-V2.xml and 29,398 bytes in size, which is incorporated herein by reference.

### REFERENCE TO A DEPOSIT OF BIOLOGICAL MATERIAL

This application contains a reference to a deposit of biological material, which deposit is incorporated herein by reference. For complete information see last paragraph of the description.

### FIELD OF THE INVENTION

The present invention relates to a method for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion to a subject. The present invention particularly relates to a method for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion by providing one or more Ligilactobacillus salivarius strains or composition comprising one or more Ligilactobacillus salivarius strains to a subject.

### BACKGROUND

A significant majority of humans exhibit abdominal discomfort on consuming dairy products due to lactose. Intestinal lactase expression is high at birth but starts to decline progressively after weaning and with age, thus eventually decreasing ability to digest dietary lactose. This failure to digest lactose in the small intestine is called lactose malabsorption (LM). The presence of mal-absorbed lactose in the colon does not necessarily result in gastrointestinal symptoms, but when this condition is related to uncomfortable manifestations such as bloating, abdominal pain and diarrhea, this leads to lactose intolerance. Up to 50% of lactose mal-absorbers are exhibiting symptoms of lactose intolerance (‘Effect of exogenous beta-galactosidase in patients with lactose malabsorption and intolerance: a crossover double-blind placebo-controlled study’ Eur J Clin Nutr 2005, 59(4):489-493, Montalto M et al. and ‘Enzyme replacement therapy for primary adult lactase deficiency. Effective reduction of lactose malabsorption and milk intolerance by direct addition of beta-galactosidase to milk at mealtime’, Gastroenterology 1984, 87(5):1072-1082, Rosado J L et al). The intensity of these gastrointestinal symptoms varies considerably depending on the degree of lactase deficiency and the presence of other gastrointestinal disorders.

As a standard practice, individuals presenting symptoms of lactose intolerance avoid dairy and dairy containing products. Avoidance of milk and milk products leads to lowering of calcium intake and consequently weakening of bones thus significantly impacting the quality of life. One way to manage lactose intolerance is to administer with exogenously produced lactase as an enzyme replacement supplementation or to take in specialized probiotics which assist in lactose digestion owing to their beta-galactosidase activity (‘A combination of acid lactase from Aspergillus oryzae and yogurt bacteria improves lactose digestion in lactose mal-digesters synergistically: A randomized, controlled, double-blind cross-over trial’ Clin Nutr 2015, 34(3):394-399, de Vrese M et al and ‘The effect of oral supplementation with Lactobacillus reuteri or tilactase in lactose intolerant patients: randomized trial’ Eur Rev Med Pharmacol Sci 2010, 14(3):163-170, Ojetti V et al.). A variety of lactase supplements or probiotics are available in the commercial space. WO08001676 A1 describes lactic acid bacteria for use as a probiotic for individuals having lactose intolerance as therapy for amelioration of lactose intolerance. US2012308524 relates to a use of composition comprising Lactobacillus delbrueckii in therapy of lactose intolerance or conditions arising from lactase deficiency. Some lactic acid bacteria which are used as probiotics in therapy of lactose intolerance either produce gas, need other sugar sources along with lactose to grow in intestines, and/or do not survive for long in the small intestine. The probiotics render relief from the gastrointestinal symptoms from smaller lactose loads while present challenges relating to providing relief from heavier dairy consumption.

Although commercially available lactases are successful to a certain extent in management of lactose intolerance, the consumers still suffer both from side effects of exogenous lactases and additionally practical downsides such as the need for adjusting the lactase dosage on every meal or dairy consumption. There are few other approaches such as microbiome re-modelling such as Galacto-oligosaccharides (GOS) approach to enhance the lactose digestion capability by intestinal flora, but these approaches have limited benefits and do not provide complete relief from abdominal discomfort.

Thus, there is a need to provide relief from lactose induced abdominal discomfort for extended hours with multiple or heavy dairy consumption through-out the day.

### SUMMARY OF THE CLAIMED INVENTION

The present invention relates to use of one or more Ligilactobacillus salivarius strains or a composition comprising such to provide relief from or prevent lactose induced abdominal discomfort; digest lactose or improve digestion of lactose; provide digestive comfort after digestion of lactose on consumption of products containing lactose; and/or provide long-lasting support and/or effect for lactose digestion to a subject.

In one embodiment, the present invention relates to Ligilactobacillus salivarius strain for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion to a subject.

In another embodiment, the present invention relates to a composition for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion of a subject, the composition comprising one or more Ligilactobacillus salivarius strains and one or more lactases.

In yet another embodiment, the present invention relates to a method for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion to a subject, the method comprising providing to a subject in need thereof an effective amount of one or more Ligilactobacillus salivarius strains or a composition comprising one or more Ligilactobacillus salivarius strains.

### OVERVIEW OF SEQUENCE LISTING

SEQ ID NO:1 is mature amino acid sequence of a GH 35 lactase isolated from Aspergillus candidus.
SEQ ID NO:2 is mature amino acid sequence of a GH 2 lactase isolated from Bifidobacterium bifidum.
SEQ ID NO:3 is mature amino acid sequence of a GH 35 lactase isolated from Penicillium cremeogriseum.
SEQ ID NO:4 is mature amino acid sequence of a GH 35 lactase isolated from Aspergillus carneus.
SEQ ID NO:5 is mature amino acid sequence of a GH 35 lactase isolated from Aspergillus wentii.
SEQ ID NO:6 is mature amino acid sequence of a GH 35 lactase isolated from Rasamsonia
byssochlamydoides.
SEQ ID NO:7 is mature amino acid sequence of a GH 35 lactase isolated from Aspergillus cervinus.
SEQ ID NO:8 is mature amino acid sequence of a GH 35 lactase isolated from Aspergillus oryzae.
SEQ ID NO:9 is mature amino acid sequence of a GH 2 lactase isolated from Lactobacillus delbrueckii subsp.
bulgaricus.
SEQ ID NO:10 is mature amino acid sequence of a GH 2 lactase isolated from Bifidobacterium bifidum.
SEQ ID NO:11 is mature amino acid sequence of a GH 2 lactase isolated from Kluyveromyces lactis.
SEQ ID NO:12 is mature amino acid sequence of a GH 2 lactase isolated from Niallia circulans.

### BRIEF DESCRIPTION OF THE DRAWINGS

A more complete understanding of the present invention may be derived by referring to the detailed description and claims when considered in connection with the Figures.

FIG. 1 demonstrates the capacity of lactose metabolization by bacterial species based on cell density and max growth rate, represented by multiple strains.

FIG. 2 represents in a pie chart and table the strains that grow to high cell densities on lactose (delta OD600>0.7) and at high max growth rate (Max growth rate<5).

FIG. 3 represents a graph that demonstrates that the vast majority of the test L. salivarius strains have superior lactose metabolization, i.e., superior cell density (delta OD600>0.7) and max growth rate (max growth rate<5).

FIG. 4 represents a graph that demonstrates the superior growth kinetics of L. salivarius on lactose as carbon source compared to L. plantarum.

FIG. 5 represents a graph that demonstrates the effect of L. salivarius DSM 34078 on reduction of lactose induced gas pressure in a human fecal slurry during fecal fermentation.

FIG. 6 represents a graph that demonstrates the effect of different L. salivarius strains on reduction of lactose induced gas pressure in a human fecal slurry during fecal fermentation.

FIG. 7 represents a graph that demonstrates proliferation of L. salivarius DSM34078 in a fecal microbial community from four different fecal donors.

FIG. 8 represents a graph that demonstrates increase in relative abundance of L. salivarius DSM34078 during fecal fermentation in three different fecal donors.

FIG. 9 represents a flow diagram of an experimental design of repeated lactose challenge.

FIG. 10 represents a graph that demonstrates survival and proliferation of L. salivarius DSM34078 in a simulated intestinal condition with repeated lactose challenge.

### DEFINITIONS

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For the sake of brevity and/or clarity, well-known functions or constructions may not be described in detail.

As used herein, the singular forms “a,” “an,” and “the” are intended to include the plural forms as well, unless the context clearly indicates otherwise.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

The term “consisting of” means including, and limited to, whatever follows the phrase “consisting of.” Thus, the phrase “consisting of” indicates that the listed elements are required or mandatory, and that no other elements may be present. The term “consisting essentially of” means including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “abdominal discomfort” is an expression for discomfort felt in the abdomen such as the feeling of pain, ache, cramp, nausea, irritation, bloating, diarrhea, flatulence or the like in the abdomen or any other symptoms associated with lactose intolerance, lactase deficiency and/or digesting lactose.

As used herein, the term “digest” means break down or help break down food in the gastrointestinal tract into substances that can be absorbed and used by the body. For purposes of the present invention, the term “digesting lactose” is breaking down of lactose in the gastrointestinal tract into monosaccharides glucose and galactose, where glucose is used as an energy source, while galactose can be utilized as precursor for glycosylation of proteins and lipids. For purposes of the present invention, the term “improving digestion of lactose” is digesting lactose for subjects who have difficulty in digesting lactose or have lactase deficiency.

As used herein, the term “digestive comfort” means absorption of lactose from gastrointestinal tract without causing any abdominal discomfort.

As used herein, the term “diarrhea” is a condition of having three or more loose or liquid stools per day, or as having more stools than is normal for that person. Acute diarrhea is defined as an abnormally frequent discharge of semisolid or fluid fecal matter from the bowel, lasting less than 14 days.

As used herein, the term “constipation” refers to infrequent bowel movements where symptoms may include hard stools, straining with bowel movements, excessive time needed to pass a bowel movement, pain with bowel movements secondary to straining, abdominal pain, abdominal bloating and/or the sensation of incomplete bowel evacuation.

As used herein, the term “abdominal bloating” is an expression for gas in abdomen that leads to a sensation that the abdomen is full or distended.

As used herein, the term “GH35 Family” refers to glycoside hydrolase family 35 that comprises enzymes with β-galactosidase activity (EC 3.2.1.23). These β-galactosidases catalyse the hydrolysis of terminal non-reducing β-D-galactose residues in, for example, lactose (1,4-O-β-D-galactopyranosyl-D-glucose), oligosaccharides, glycolipids, and glycoproteins. GH35 β-galactosidases demonstrate specificity towards β-1,3-, β-1,6- or β-1,4-galactosidic linkages, and are most active under acidic conditions. Lactases belong to the β-galactosidase family of enzymes.

As used herein, the term “GH2 Family” refers to glycoside hydrolase family 2 that comprises the enzymes glycoside hydrolases (EC 3.2.1). The GH2 Family comprises enzymes with several known activities e.g., beta-galactosidase (EC 3.2.1.23); beta-mannosidase (EC 3.2.1.25); beta-glucuronidase (EC 3.2.1.31). For the purposes of the present invention, GH2 Family comprises enzymes with beta-galactosidase (EC 3.2.1.23) activities. Lactases belong to β-galactosidase family of enzymes.

As used herein, the term “acid lactase” means a lactase having optimum activity and/or stability at a pH range between pH 3.5 and pH 5.0.

As used herein, the term “neutral lactase” means a lactase having optimum activity and/or stability at a pH range between pH 6 and pH 9, preferably between pH 7 and pH 8, more preferably between pH 6.5 and pH 7.5.

As used herein, the term “fungal lactase” means a lactase derived from the fungus, preferably derived from Aspergillus oryzae.

As used herein, the term “mature polypeptide” means a polypeptide in its mature form following N terminal and/or C-terminal processing (e.g., removal of signal peptide).

As used herein, the term “Ligilactobacillus salivarius” describes a rod-shaped, gram-positive bacteria.

Ligilactobacillus salivarius was previously designated “Lactobacillus salivarius”, a term that is still often used. “Ligilactobacillus salivarius” may be used interchangeably with “L. salivarius” and “Lactobacillus salivarius”.

As used herein, the term “maximum growth rate” is used to measure the growth rate of bacteria which means bacterial growth parameter calculated in milli-optical density per minute based on the inflection point of the slope of the growth curve in the exponential phase. For the purposes of the present invention, the maximum growth rate is used to identify the Ligilactobacillus strains which have higher growth rate in presence of lactose as a single carbon source in a growth medium.

As used herein, “lactose medium” is a selective growth medium which has lactose as the carbon source and other essential nutrients required for bacterial growth.

As used herein, “gas pressure measurement system” is a technique to measure gas pressure in closed vessels with a sensor for continuously measuring gas pressure within an observation period. The closed vessels can be Hungate tubes with a rubber septum (originally for cultivation of anaerobic microorganisms). For the purposes of the present invention, the gas pressure measurement system is used to measure the amount of gas generated in fecal samples with and without Ligilactobacillus salivarius strains. The terms “gas pressure development” and “gas generation” are herein used interchangeably.

As used herein, “delta OD600” is a reference to a spectrometer method that is used to help estimate the concentration or “number of cells per volume” of bacteria or other cells within a liquid sample, where OD 600 is reference to the 600-nanometer (nm) wavelength used to measure optical density of the liquid sample. For purposes of the present invention,

delta OD 600=(final OD600 with lactose as the main carbon source in a sample)−(final OD600 without lactose in a sample).

As used herein, “sequence identity” is the relatedness between two amino acid sequences or between two nucleotide sequences. is described by the parameter “sequence identity”. For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

While certain embodiments of the present disclosure will hereinafter be described with reference to embodiments thereof, it will be understood by those of ordinary skill in the art that various changes in form and details may be made therein without departing from the spirit and scope of the present invention as defined by the claims.

### DETAILED DESCRIPTION OF THE INVENTION

The present invention relates to uses, strains, composition and methods for providing relief or preventing from lactose induced abdominal discomfort; digesting lactose or improving digestion of lactose; providing digestive comfort after digestion of lactose on consumption of products containing lactose; and/or providing long-lasting support and/or effect for lactose digestion to a subject.

The inventors identified with the present invention Ligilactobacillus salivarius strains which when provided to a human, do not produce gas after digestion of lactose, are able to grow on lactose as single carbon source at very high rate and are able to survive in gastrointestinal tract, specifically in small intestinal conditions. In some embodiments, Ligilactobacillus salivarius is referred to as Lactobacillus salivarius. The present invention provides Ligilactobacillus salivarius strains which provide relief from discomfort caused to humans who are lactose intolerant and/or people who have difficulty in digesting lactose. The present invention provides Ligilactobacillus salivarius strains which prevent lactose induced abdominal discomfort of a human. Further, the present invention provides Ligilactobacillus salivarius strains which digest lactose or improves digestion of lactose in humans who are lactose intolerant, have difficulty in digesting lactose and/or have lactase deficiency. The present invention provides Ligilactobacillus salivarius strains which provide digestive comfort by digesting lactose on consumption of products containing lactose. The present invention provides Ligilactobacillus salivarius strains which have long-lasting support and/or effect for lactose digestion in a subject.

In one embodiment of the invention, one or more Ligilactobacillus salivarius strains are provided to a human in combination with one or more lactases. In order to provide relief from lactose induced abdominal discomfort for extended hours and dairy consumption multiple times through-out the day, the inventors surprisingly identified the power of instant lactose digestion by one or more lactases and extended action by one or more Ligilactobacillus salivarius strains, which are efficient in digesting lactose. This dual action concept may be used to efficiently tackle lactose intolerance related gastrointestinal symptoms by combining the exogenous lactase with the endogenous lactase activity of Ligilactobacillus salivarius strains in a dosage form. One embodiment of the present invention thus covers a dosage form comprising one or more Ligilactobacillus salivarius strains and one or more lactases. The combination of exogenous lactase and Ligilactobacillus salivarius strains of the present invention, when consumed, ensures that the consumer has several hours of protection from gastrointestinal symptoms over repeated dairy consumption throughout the day. On consumption of the dosage form with the lactose containing meal, the lactase ensures immediate hydrolysis of incoming dietary lactose while the Ligilactobacillus salivarius strains of the present invention on the other hand enables lactose digestion further during the day on additional lactose consumption. Thus, the mode-of-action of the combination delivers protection to consumers from dietary lactose induced gastrointestinal symptoms and manages lactose intolerance effectively improving the quality of life. The present invention provides a prolonged relief from lactose induced abdominal discomfort in humans affected by and/or intolerant to lactose, having difficulty in digesting lactose and/or having lactase deficiency.

Ligilactobacillus salivarius Strains

The one or more Ligilactobacillus salivarius strains of the invention or for use in the present invention may be any Ligilactobacillus salivarius strains that digest lactose. The level of digestion may e.g., be determined by measuring the maximum growth rate of the Ligilactobacillus salivarius strains on a lactose medium and/or by measuring the delta OD600 value in a lactose medium. In one embodiment, when Ligilactobacillus salivarius strains of the invention or of use in the invention are grown on a lactose medium, the maximum growth rate of the Ligilactobacillus salivarius strains is at least 5.0 mean optical density/minute in a lactose medium. In another or further embodiment, the Ligilactobacillus salivarius strains have a delta OD600 value of at least 0.7 in a lactose medium. The lactose medium may e.g., be Yeast Lactose Acetate (YLA) medium. The ingredients of the YLA medium are listed in Table 1 below:

TABLE 1

| Substance | Grams/Liter (g/L) | |
|---|---|---|
| Yeast extract X-Seed peptone | 25.0 | g |
| Alpha-Lactose-Monohydrate | 20.0 | g |
| Tween 80 | 1.0 | g |
| Diammonium Hydrogen Citrate | 2.0 | g |
| Sodium Acetate | 5.0 | g |
| Magnesium Sulfate Heptahydrate | 0.1 | g |
| Manganese (II)Sulfate Monohydrate | 0.05 | g |
| Dipotassium Phosphate | 2.0 | g |

In one embodiment, the Ligilactobacillus salivarius strains of the invention or for use in the invention survive in the gastrointestinal tract of humans. In one embodiment, the Ligilactobacillus salivarius strains survive in the gastrointestinal tract for at least 4 hours. In one embodiment, the Ligilactobacillus salivarius strains grow on lactose in vitro and/or in the gastrointestinal tract of humans. In another or further embodiment, the Ligilactobacillus salivarius strains grow on lactose, preferably grow on lactose as the major carbon source, such as e.g., grow on lactose as a single carbon source. In one embodiment, the Ligilactobacillus salivarius strains prefer to grow on lactose as a carbon source. That the Ligilactobacillus salivarius strains ‘prefer to grow’ on lactose means the Ligilactobacillus salivarius strains grow faster on lactose compared to other sugars, and the Ligilactobacillus salivarius strains first break down lactose compared to all other available sugar carbon sources. In one embodiment, the Ligilactobacillus salivarius strains according to the invention or for use in the invention reduce gas production of fecal matter comprising lactose in a gas pressure measurement system. In an embodiment, the Ligilactobacillus salivarius strains of the invention or for the use in the invention reduce at least 20% gas pressure development by microbiota. In another embodiment, the Ligilactobacillus salivarius strains of the invention or for the use in the invention reduce up to 60% gas pressure development by microbiota. In a preferred embodiment, the Ligilactobacillus salivarius strains of the invention or for use in the invention reduce at least 30%, such as at least 40% or at least 50% gas pressure development by microbiota. In an embodiment, the Ligilactobacillus salivarius strains express one or more lactases. In another embodiment, the amount of lactase expressed from a Ligilactobacillus salivarius strain is measured in vitro based on the amount of lactose reduced from a lactose medium on which the Ligilactobacillus salivarius strain is grown. The amount of lactose reduced from the lactose medium is in an embodiment at least 30%, such as at least 40%, 50%, 60%, 70% or 75%.

In an embodiment of the present invention, the one or more Ligilactobacillus salivarius strains of the present invention and for use, for a method and for a composition of the present invention is Ligilactobacillus salivarius strain having deposit number DSM 34078. In another embodiment of the present invention, the one or more Ligilactobacillus salivarius strains is Ligilactobacillus salivarius strain having all the identifying characteristics of Ligilactobacillus salivarius strain having deposit number DSM 34078, or mutant thereof.

### Lactase

In an embodiment of the present invention, the one or more lactases for use in combination with one or more Ligilactobacillus salivarius strains belong to the GH35 family or the GH2 family. In an embodiment of the present invention, the one or more lactases are acid lactases and/or neutral lactases. In an embodiment of the present invention, the one or more lactases are fungal lactases.

In a further embodiment, the one or more lactases is a polypeptide selected from the group consisting of:
(a) a polypeptide having at least 80% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12;
(b) a polypeptide derived from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12 by having 1-30 alterations (e.g., substitutions, deletions and/or insertions) at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
(c) a polypeptide derived from the polypeptide of (a) or (b), wherein the N- and/or C-terminal end has been extended by addition of one or more amino acids, and
(d) a fragment of the polypeptide of (a), (b) or (c).

In another embodiment, the polypeptide has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the polypeptide sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12.

In a preferred embodiment of the present invention, the lactase is a polypeptide comprising, consisting essentially of, or consisting of SEQ ID NO:1. In another preferred embodiment of the present invention, the lactase is a polypeptide comprising, consisting essentially of, or consisting of SEQ ID NO:2.

In an embodiment, the polypeptide may have an N-terminal and/or C-terminal extension of one or more amino acids, e.g., 1-5 amino acids.

In one embodiment, the polypeptide is derived from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12 by substitution, deletion or addition of one or several amino acids. In some embodiments, the polypeptide is a variant of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12 comprising a substitution, deletion, and/or insertion at one or more positions. In one embodiment, the number of amino acid substitutions, deletions and/or insertions introduced into the polypeptide of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

### Composition

In one embodiment, the present invention relates to a composition for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion of a subject, wherein the composition comprises one or more Ligilactobacillus salivarius strains. In a further embodiment, the composition further comprises one or more lactases. In an embodiment of the composition, the dosage form of the composition can be a single dosage form. In an embodiment of the composition, the composition can be in a dosage form such as, but not limited to, capsules, powders, microcapsules, tablets, dragees, pellets and granules. In an embodiment of the composition, the amount of the one or more Ligilactobacillus salivarius strains in the composition is in a range of 1E+08 Colony Forming Unit (CFU) to 1E+11 CFU. In a further embodiment of the composition, the amount of the one or more Ligilactobacillus salivarius strains in the composition is in a range of 1E+09 Colony Forming Unit (CFU) to 1E+11 CFU.

In a preferred embodiment, the amount of the one or more Ligilactobacillus salivarius strains in the composition is about 1E+09 CFU. In an embodiment of the composition, the amount of the one or more lactases is at least 4500 Food Chemicals Codex (FCC) units. In a preferred embodiment, the amount of the one or more lactases is about 10000 FCC units.

In an embodiment where the composition of the invention comprises one or more lactases, the lactase(s) are immediately available for lactose breakdown in the gastrointestinal tract. Ligilactobacillus salivarius strains of the composition release lactase slowly in the gastrointestinal tract and the released lactase is available in the gastrointestinal tract throughout the day. The lactase released by Ligilactobacillus salivarius strains are available to breakdown lactose consumed by the subject anytime throughout the day. In an embodiment of the composition, the one or more lactases of the composition break down lactose that the subject consumes immediately after consumption of the composition and the one or more Ligilactobacillus salivarius strains break down lactose that the subject consumes for a time period of at least up to 4 hours after consumption of the composition, preferably that the subject consumes throughout the day. “Immediately” is here understood as the lactase breaks down lactose as soon as it is in contact with the lactose. For example, if the subject consumes milk or any dairy products immediately after consumption of a composition comprising one or more Ligilactobacillus salivarius strains and one or more lactases, the lactases in the composition break down the lactose from the consumed milk or dairy products as soon as in contact with the lactose, whereas the Ligilactobacillus salivarius strains break down the lactose from milk or dairy products consumed anytime throughout the day. In one embodiment, the one or more Ligilactobacillus salivarius strains break down lactose that the subject consumes for a time period of at least up to 8 hours after consumption of the composition. In another embodiment, the one or more Ligilactobacillus salivarius strains break down lactose that the subject consumes for a time period of at least up to 12 hours after consumption of the composition. In yet another embodiment, the one or more Ligilactobacillus salivarius strains break down lactose that the subject consumes for a time period of at least up to 18 hours after consumption of the composition. The composition may be provided to the subject 1, 2 or 3 times in a day to provide relief from or prevent lactose induced abdominal discomfort; digest lactose or improve digestion of lactose; provide digestive comfort after digestion of lactose on consumption of products containing lactose; and/or provide long-lasting support and/or effect for lactose digestion of a subject throughout the day. In one embodiment, the composition is provided to the subject two times per day to provide relief from or prevent lactose induced abdominal discomfort; digest lactose or improve digestion of lactose; provide digestive comfort after digestion of lactose on consumption of products containing lactose; and/or provide long-lasting support and/or effect for lactose digestion of a subject throughout the day. In a preferred embodiment, the composition may be provided to the subject once in a day to provide relief from or prevent lactose induced abdominal discomfort; digest lactose or improve digestion of lactose; provide digestive comfort after digestion of lactose on consumption of products containing lactose; and/or provide long-lasting support and/or effect for lactose digestion of a subject throughout the day.

### Uses and Methods

Also disclosed herein is the use of one or more Ligilactobacillus salivarius strains or a composition comprising such, for a use which is selected from a group consisting of:
a. providing relief from or preventing lactose induced abdominal discomfort,
b. digesting lactose or improving digestion of lactose;
c. providing digestive comfort after digestion of lactose on consumption of products containing lactose;
d. providing long-lasting support and/or effect for lactose digestion; and
e. any combination thereof.

In one embodiment, the use is providing relief from or preventing lactose induced abdominal discomfort. In one embodiment, the use is digesting lactose or improving digestion of lactose. In one embodiment, the use is providing digestive comfort after digestion of lactose on consumption of products containing lactose. In one embodiment, the use is providing long-lasting support and/or effect for lactose digestion.

In an embodiment of the use, the effect of the one or more Ligilactobacillus salivarius strains or the composition comprising such, after consumption, is for at least up to 4 hours on a subject, preferably for at least up to 8 hours. In another embodiment, the effect of the one or more Ligilactobacillus salivarius strains or the composition comprising such after consumption is for at least up to 12 hours, 18 hours or 24 hours. In an embodiment, the Ligilactobacillus salivarius strains or composition are provided 1, 2 or 3 times daily to a subject, preferably at least once daily to a subject.

In an embodiment of the use, the composition further comprises one or more lactases. In another embodiment of the use, the one or more lactases break down lactose that the subject consumes immediately after consumption of the composition and the one or more Ligilactobacillus salivarius strains break down lactose that the subject consumes for a time period of at least up to 4 hours after consumption of the composition, preferably that the subject consumes for a time period of up to 24 hours after consumption of the composition. In another embodiment, the one or more Ligilactobacillus salivarius strains breaks down lactose that the subject consumes for a time period of at least up to 8 hours after consumption of the composition. In another embodiment, the one or more Ligilactobacillus salivarius strains breaks down lactose that the subject consumes for a time period of at least up to 12 hours, 18 hours or 24 hours after consumption of the composition.

In another embodiment, the present invention relates to a method for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion to a subject; the method comprising providing to a subject in need thereof an effective amount of one or more Ligilactobacillus salivarius strains or a composition comprising one or more Ligilactobacillus salivarius strains.

In one embodiment, the method is for providing relief from or preventing lactose induced abdominal discomfort. In one embodiment, the method is for digesting lactose or improving digestion of lactose. In one embodiment, the method is for providing digestive comfort after digestion of lactose on consumption of products containing lactose. In one embodiment, the method is for providing long-lasting support and/or effect for lactose digestion.

In an embodiment of the method, the one or more Ligilactobacillus salivarius strains or a composition comprising one or more Ligilactobacillus salivarius strains is provided 1, 2 or 3 times daily to a subject, preferably at least once daily to the subject. In a preferred embodiment of the method, the one or more Ligilactobacillus salivarius strains or a composition comprising one or more Ligilactobacillus salivarius strains is provided orally.

In an embodiment of the method, the composition further comprises one or more lactases. In another embodiment of the method, the one or more lactases break down lactose that the subject consumes immediately after consumption of the composition and the one or more Ligilactobacillus salivarius strains break down lactose that the subject consumes for a time period of at least up to 4 hours after consumption of the composition, preferably that the subject consumes for a time period of up to 24 hours after consumption of the composition. In another embodiment, the one or more Ligilactobacillus salivarius strains breaks down lactose that the subject consumes for a time period of at least up to 8 hours after consumption of the composition. In another embodiment, the one or more Ligilactobacillus salivarius strains breaks down lactose that the subject consumes for a time period of at least up to 12 hours, 18 hours or 24 hours after consumption of the composition.

Particular embodiments of the present disclosure are described in the following numbered paragraphs:

1. Use of one or more Ligilactobacillus salivarius strains or a composition comprising such, for a use which is selected from a group consisting of:
a. providing relief from or preventing lactose induced abdominal discomfort;
b. digesting lactose or improving digestion of lactose;
c. providing digestive comfort after digestion of lactose on consumption of products containing lactose;
d. providing long-lasting support and/or effect for lactose digestion; and
e. any combination thereof.

2. The use according to paragraph 1, wherein the effect of the one or more Ligilactobacillus salivarius strains or the composition comprising such, after consumption, is for a time period of at least up to 4 hours, preferably for at least up to 8 hours.

3. The use according to paragraph 1, wherein the effect of the one or more Ligilactobacillus salivarius strains or the composition comprising such, after consumption, is for a time period of up to 24 hours.

4. The use according to any one of paragraphs 1 or 2, wherein the lactose induced abdominal discomfort is diarrhea, constipation, bloating, pain, flatulence, nausea, and/or gas induced from ingestion of products comprising lactose.

5. The use according to any one of the preceding paragraphs, wherein the Ligilactobacillus salivarius strains or composition are provided from 1 to 3 times daily to a subject, preferably at least once daily to a subject.

6. The use according to any one of the preceding paragraphs, wherein the one or more Ligilactobacillus salivarius strains or the composition is provided orally to the subject.

7. The use according to any one of the preceding paragraphs, wherein the Ligilactobacillus salivarius strain expresses one or more lactases.

8. The use according to any one of the preceding paragraphs, wherein the amount of lactases expressed from the Ligilactobacillus salivarius strain is measured in vitro based on the amount of lactose reduced from a lactose medium on which the Ligilactobacillus salivarius strain is grown.

9. The use according to any one of the preceding paragraphs, wherein the amount of lactose reduced from the lactose medium is at least 30%, preferably 75%.

10. The use according to any one of the preceding paragraphs, wherein the amount of lactose reduced from the lactose medium is at least 50% in a time period of up to 24 hours, preferably 75%.

11. The use according to any one of the preceding paragraphs, wherein the Ligilactobacillus salivarius strain has a deposit accession number DSM 34078, or the Ligilactobacillus salivarius strain is Ligilactobacillus salivarius strain having all the identifying characteristics of Ligilactobacillus salivarius strain having a deposit accession number DSM 34078, or a mutant thereof.

12. The use according to any one of the preceding paragraphs, wherein the Ligilactobacillus salivarius strains have the maximum growth rate of at least 5.0 mean optical density/minute in a lactose medium.

13. The use according to any one of the preceding paragraphs, wherein the Ligilactobacillus salivarius strains have a delta OD600 value of at least 0.7 in a lactose medium.

14. The use according to any one of the preceding paragraphs, wherein the Ligilactobacillus salivarius strains have the maximum growth rate of at least 5.0 mean optical density/minute in a lactose medium and have a delta OD600 value of at least 0.7 in a lactose medium.

15. The use according to any one of the preceding paragraphs, wherein the Ligilactobacillus salivarius strains survive in the gastrointestinal tract.

16. The use according to any one of the preceding paragraphs, wherein the Ligilactobacillus salivarius strains survive in the gastrointestinal tract for at least 4 hours.

17. The use according to any one of the preceding paragraphs, wherein the Ligilactobacillus salivarius strains grow on lactose in vitro and/or in the gastrointestinal tract of a subject.

18. The use according to any one of the preceding paragraphs, wherein the Ligilactobacillus salivarius strains prefer to grow on lactose in the presence of other sugars.

19. The use according to any one of the preceding paragraphs, wherein the Ligilactobacillus salivarius strains reduce gas production of fecal matter comprising lactose in a gas pressure measurement system.

20. The use according to any one of the preceding paragraphs, wherein the amount of the Ligilactobacillus salivarius strains is in a range of 1E+08 Colony Forming Unit (CFU) to 1E+11 CFU.

21. The use according to any one of the preceding paragraphs, wherein the amount of the Ligilactobacillus salivarius strains is in a range of 1E+09 CFU to 1E+11 CFU, preferably in a range of 1E+09 CFU to 1E+10 CFU, more preferably about 1E+09 CFU.

22. The use according to any one of the preceding paragraphs, wherein the composition further comprises one or more lactases.

23. The use according to paragraph 22, wherein the one or more lactases belong to the GH35 Family or GH2 Family.

24. The use according to any one of paragraphs 22 or 23, wherein the one or more lactases are acid lactases, and/or neutral lactases.

25. The use according to any one of paragraphs 22-24, wherein the one or more lactases are fungal lactases.

26. The use according to any one of paragraphs 22-25, wherein the one or more lactases is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12;
b. a polypeptide derived from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).

27. The use according to any one of paragraphs 22-26, wherein the lactase is a polypeptide comprising, consisting essentially of, or consisting of SEQ ID NO:1.

28. The use according to any one of paragraphs 22-26, wherein the lactase is a polypeptide comprising, consisting essentially of, or consisting of SEQ ID NO:2.

29. The use according to any one of paragraphs 22-28, wherein the amount of the lactases is at least 4500 Food Chemicals Codex (FCC) units.

30. The use according to any one of paragraphs 22-29, wherein the amount of the one or more lactases is 10000 FCC units.

31. The use according to any one of paragraphs 22-30, wherein the one or more lactases is for fast acting support and/or effect on lactose, and one or more Ligilactobacillus salivarius strains is for long lasting support and/or effect on lactose.

32. The use according to any one of paragraphs 22-31, wherein the one or more lactases breaks down lactose that the subject consumes immediately after consumption of the composition and the one or more Ligilactobacillus salivarius strains breaks down lactose that the subject consumes for a time period of at least up to 4 hours after consumption of the composition, preferably lactose that the subject consumes for a time period of up to 24 hours after consumption of the composition.

33. The use according to any one of paragraphs 22-32, wherein the one or more Ligilactobacillus salivarius strains breaks down lactose that the subject consumes for a time period of at least up to 8 hours after consumption of the composition.

34. The use according to any one of paragraphs 22-33, wherein the one or more Ligilactobacillus salivarius strains breaks down lactose that the subject consumes for a time period of at least up to 12 hours after consumption of the composition.

35. One or more Ligilactobacillus salivarius strains or a composition comprising such for a use which is selected from a group consisting of:
a. for providing relief from or preventing lactose induced abdominal discomfort;
b. for digesting lactose or improving digestion of lactose;
c. for providing digestive comfort after digestion of lactose on consumption of products containing lactose;
d. for providing long-lasting support and/or effect for lactose digestion; and
e. for any combination thereof.

36. The one or more Ligilactobacillus salivarius strains or a composition comprising such for a use according to paragraph 35, wherein the effect of the one or more Ligilactobacillus salivarius strains or the composition comprising such, after consumption, is for a time period of at least up to 4 hours, preferably for at least up to 8 hours.

37. The one or more Ligilactobacillus salivarius strains or a composition comprising such for a use according to any one of paragraphs 35 or 36, wherein the effect of the one or more Ligilactobacillus salivarius strains or the composition comprising such, after consumption, is for a time period of up to 24 hours.

38. The one or more Ligilactobacillus salivarius strains or a composition comprising such for a use according to any one of paragraphs 35-37, wherein lactose induced abdominal discomfort is diarrhea, constipation, bloating and/or gas induced from ingestion of products comprising lactose.

39. The one or more Ligilactobacillus salivarius strains or a composition comprising such for a use according to any one of paragraphs 35-38, wherein the Ligilactobacillus salivarius strains or composition are provided from 1 to 3 times daily to a subject, preferably at least once daily to a subject.

40. The one or more Ligilactobacillus salivarius strains or a composition comprising such for a use according to any one of paragraphs 35-39, wherein the one or more Ligilactobacillus salivarius strains or the composition is provided orally to the subject

41. The one or more Ligilactobacillus salivarius strains or a composition comprising such for a use according to any one of paragraphs 35-40, wherein the Ligilactobacillus salivarius strain expresses one or more lactases.

42. The one or more Ligilactobacillus salivarius strains or a composition comprising such for a use according to any one of paragraphs 35-41, wherein the amount of lactases expressed from the Ligilactobacillus salivarius strain is measured in vitro based on the amount of lactose reduced from a lactose medium on which the Ligilactobacillus salivarius strain is grown.

43. The one or more Ligilactobacillus salivarius strains or a composition comprising such for a use according to any one of paragraphs 35-42, wherein the amount of lactose reduced from the lactose medium is at least 30%, preferably 75%.

44. The one or more Ligilactobacillus salivarius strains or a composition comprising such for a use according to any one of paragraphs 35-43, wherein the amount of lactose reduced from the lactose medium is at least 50% in a time period of up to 24 hours, preferably 75%.

45. The one or more Ligilactobacillus salivarius strains or a composition comprising such for a use according to any one of paragraphs 35-44, wherein the Ligilactobacillus salivarius strain has a deposit accession number DSM 34078, or the Ligilactobacillus salivarius strain is Ligilactobacillus salivarius strain having all the identifying characteristics of Ligilactobacillus salivarius strain having a deposit accession number DSM 34078, or a mutant thereof.

46. The one or more Ligilactobacillus salivarius strains or a composition comprising such for a use according to any one of paragraphs 35-45, wherein the Ligilactobacillus salivarius strains have the maximum growth rate of at least 5.0 mean optical density/minute in a lactose medium.

47. The one or more Ligilactobacillus salivarius strains or a composition comprising such for a use according to any one of paragraphs 35-46, wherein the Ligilactobacillus salivarius strains have a delta OD600 value of at least 0.7 in a lactose medium.

48. The one or more Ligilactobacillus salivarius strains or a composition comprising such for a use according to any one of paragraphs 35-47, wherein the Ligilactobacillus salivarius strains have the maximum growth rate of at least 5.0 mean optical density/minute in a lactose medium and have a delta OD600 value of at least 0.7 in a lactose medium.

49. The one or more Ligilactobacillus salivarius strains or a composition comprising such for a use according to any one of paragraphs 35-48, wherein the Ligilactobacillus salivarius strains survive in the gastrointestinal tract.

50. The one or more Ligilactobacillus salivarius strains or a composition comprising such for a use according to any one of paragraphs 35-49, wherein the Ligilactobacillus salivarius strains survive in the gastrointestinal tract for at least 4 hours.

51. The one or more Ligilactobacillus salivarius strains or a composition comprising such for a use according to any one of paragraphs 35-50, wherein the Ligilactobacillus salivarius strains grow on lactose in vitro and/or in the gastrointestinal tract of a subject.

52. The one or more Ligilactobacillus salivarius strains or a composition comprising such for a use according to any one of paragraphs 35-51, wherein the Ligilactobacillus salivarius strains prefer to grow on lactose in the presence of other sugars.

53. The one or more Ligilactobacillus salivarius strains or a composition comprising such for a use according to any one of paragraphs 35-52, wherein the Ligilactobacillus salivarius strains reduce gas production of fecal matter comprising lactose in a gas pressure measurement system.

54. The one or more Ligilactobacillus salivarius strains or a composition comprising such for a use according to any one of paragraphs 35-53, wherein the amount of the one or more Ligilactobacillus salivarius strains is in a range of 1E+08 Colony Forming Unit (CFU) to 1E+11 CFU, preferably in a range of 1E+09 CFU to 1E+11 CFU, preferably in the range of 1E+09 CFU to 1E+10 CFU, more preferably about 1E+09 CFU.

55. The one or more Ligilactobacillus salivarius strains or a composition comprising such for a use according to any one of paragraphs 35-54, wherein the composition further comprises one or more lactases.

56. The one or more Ligilactobacillus salivarius strains or a composition comprising such for a use according to paragraph 55, wherein the one or more lactases belong to the GH35 Family or GH2 Family.

57. The one or more Ligilactobacillus salivarius strains or a composition comprising such for a use according to paragraph 55 or 56, wherein the one or more lactases are acid lactases, and/or neutral lactases.

58. The one or more Ligilactobacillus salivarius strains or a composition comprising such for a use according to any one of paragraphs 55-57, wherein the one or more lactases are fungal lactases.

59. The one or more Ligilactobacillus salivarius strains or a composition comprising such for a use according to any one of paragraphs 55-58, wherein the one or more lactases is a polypeptide selected from the group consisting of:
e. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12;
f. a polypeptide derived from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
g. a polypeptide derived from the polypeptide of (a) or (b), wherein the N- and/or C-terminal end has been extended by addition of one or more amino acids, and
h. a fragment of the polypeptide of (a), (b), or (c).

60. The one or more Ligilactobacillus salivarius strains or a composition comprising such for a use according to any one of paragraphs 55-59, wherein the lactase is a polypeptide comprising, consisting essentially of, or consisting of SEQ ID NO:1.

61. The one or more Ligilactobacillus salivarius strains or a composition comprising such for a use according to any one of paragraphs 55-59, wherein the lactase is a polypeptide comprising, consisting essentially of, or consisting of SEQ ID NO:2.

62. The one or more Ligilactobacillus salivarius strains or a composition comprising such for a use according to any one of paragraphs 55-61, wherein the amount of the one or more lactases is at least 4500 Food Chemicals Codex (FCC) units.

63. The one or more Ligilactobacillus salivarius strains or a composition comprising such for a use according to any one of paragraphs 55-62, wherein the amount of the one or more lactases is 10000 FCC units.

64. The one or more Ligilactobacillus salivarius strains or a composition comprising such for a use according to any one of paragraphs 55-63, wherein the one or more lactases is for fast acting support and/or effect on lactose, and one or more Ligilactobacillus salivarius strains is for long lasting support and/or effect on lactose.

65. The one or more Ligilactobacillus salivarius strains or a composition comprising such for a use according to any one of paragraphs 55-64, wherein the one or more lactases breaks down lactose that the subject consumes immediately after consumption of the composition and the one or more Ligilactobacillus salivarius strains breaks down lactose that the subject consumes for a time period of at least up to 4 hours after consumption of the composition, preferably lactose that the subject consumes for a time period of up to 24 hours after consumption of the composition.

66. The one or more Ligilactobacillus salivarius strains or a composition comprising such for a use according to any one of paragraphs 55-65, wherein the one or more Ligilactobacillus salivarius strains breaks down lactose that the subject consumes for a time period of at least up to 8 hours after consumption of the composition.

67. The one or more Ligilactobacillus salivarius strains or a composition comprising such for a use according to any one of paragraphs 55-66, wherein the one or more Ligilactobacillus salivarius strains breaks down lactose that the subject consumes for a time period of at least up to 12 hours after consumption of the composition.

68. The one or more Ligilactobacillus salivarius strains or a composition comprising such for a use according to any one of paragraphs 55-67, wherein the amount of the one or more Ligilactobacillus salivarius strains is in a range of 1E+08 CFU to 1E+11 CFU, preferably in a range of 1E+09 CFU to 1E+11 CFU, preferably in the range of 1E+09 CFU to 1E 10 CFU, more preferably about 1E+09 CFU.

69. The one or more Ligilactobacillus salivarius strains or a composition comprising such for a use according to any one of paragraphs 54-67, wherein the composition further comprises one or more lactases.

70. A Ligilactobacillus salivarius strain having a deposit number DSM 34078 or a Ligilactobacillus salivarius strain having all the identifying characteristics of Ligilactobacillus salivarius strain having a deposit accession number DSM 34078, or mutant thereof.

71. The Ligilactobacillus salivarius strain according to paragraph 70, wherein the Ligilactobacillus salivarius strain survives in gastrointestinal tracts.

72. The Ligilactobacillus salivarius strain according to any one of paragraphs 70 or 71, wherein the Ligilactobacillus salivarius strain grows on lactose in vitro and/or in the gastrointestinal tract of a subject.

73. The Ligilactobacillus salivarius strain according to any one of paragraphs 70-72, wherein the Ligilactobacillus salivarius strain prefers to grow on lactose in presence of other sugars.

74. The Ligilactobacillus salivarius strain according to any one of paragraphs 70-73, wherein the Ligilactobacillus salivarius strain reduces gas production of fecal matter comprising lactose in a gas pressure measurement system.

75. The Ligilactobacillus salivarius strain according to any one of paragraphs 70-74, wherein the Ligilactobacillus salivarius strain expresses one or more lactases.

76. The Ligilactobacillus salivarius strain according to any one of paragraphs 70-75, wherein the amount of lactases expressed from the Ligilactobacillus salivarius strain is measured in vitro based on the amount of lactose reduced from a lactose medium on which the Ligilactobacillus salivarius strain is grown.

77. The Ligilactobacillus salivarius strain according to any one of paragraphs 70-76, wherein the amount of lactose reduced from the lactose medium is at least 50%, preferable 75%.

78. A composition for providing relief from or preventing lactose induced abdominal discomfort; digesting lactose or improving digestion of lactose; providing digestive comfort after digestion of lactose on consumption of products containing lactose; and/or providing long-lasting support and/or effect for lactose digestion of a subject, the composition comprising:
(a) one or more Ligilactobacillus salivarius strains; and
(b) one or more lactases.

79. The composition according to paragraph 78, wherein the effect of the one or more Ligilactobacillus salivarius strains or the composition comprising such, after consumption, is for a time period of at least up to 4 hours, preferably for at least up to 8 hours.

80. The composition according to paragraph 78 or 79, wherein the effect of the one or more Ligilactobacillus salivarius strains or the composition comprising such, after consumption, is for a time period of up to 24 hours.

81. The composition according to paragraph 78-80, wherein lactose induced abdominal discomfort is diarrhea, constipation, bloating and/or gas induced from ingestion of products comprising lactose.

82. The composition according to any one of paragraphs 78-81, wherein the Ligilactobacillus salivarius strains or composition are provided from 1 to 3 times daily to a subject, preferably at least once daily to a subject.

83. The composition according to any one of paragraphs 78-82, wherein the one or more Ligilactobacillus salivarius strains or the composition is provided orally to the subject.

84. The composition according to any one of paragraphs 78-83, wherein the Ligilactobacillus salivarius strain expresses one or more lactases.

85. The composition according to any one of paragraphs 78-84, wherein the amount of lactases expressed from the Ligilactobacillus salivarius strain is measured in vitro based on the amount of lactose reduced from a lactose medium on which the Ligilactobacillus salivarius strain is grown.

86. The composition according to any one of paragraphs 78-85, wherein the amount of lactose reduced from the lactose medium is at least 30%, preferably 75%.

87. The composition according to any one of paragraphs 78-86, wherein the amount of lactose reduced from the lactose medium is at least 50% in a time period of up to 24 hours, preferably 75%.

88. The composition according to paragraph 78-87, wherein the Ligilactobacillus salivarius strain has a deposit accession number DSM 34078, or the Ligilactobacillus salivarius strain is Ligilactobacillus salivarius strain having all the identifying characteristics of Ligilactobacillus salivarius strain having a deposit accession number DSM 34078, or mutant thereof.

89. The composition according to any one of paragraphs 78-88, wherein the Ligilactobacillus salivarius strains have a maximum growth rate of at least 5.0 mean optical density/minute in a lactose medium.

90. The composition according to any one of paragraphs 78-89, wherein the Ligilactobacillus salivarius strains have a delta OD600 value of at least 0.7 in a lactose medium.

91. The composition according to any one of paragraphs 78-90, wherein the Ligilactobacillus salivarius strains have a maximum growth rate of at least 5.0 mean optical density/minute in a lactose medium and have a delta OD600 value of at least 0.7 in a lactose medium.

92. The composition according to any one of paragraphs 78-91, wherein the Ligilactobacillus salivarius strains survive in the gastrointestinal tract.

93. The composition according to any one of paragraphs 78-92, wherein the Ligilactobacillus salivarius strains survive in the gastrointestinal tract for at least 4 hours.

94. The composition according to any one of paragraphs 78-93, wherein the Ligilactobacillus salivarius strains grow on lactose in vitro and/or in the gastrointestinal tract of a subject.

95. The composition according to paragraph 78-94, wherein the Ligilactobacillus salivarius strains prefer to grow on lactose in the presence of other sugars.

96. The composition according to any one of paragraphs 78-95, wherein the Ligilactobacillus salivarius strains reduce gas production of fecal matter comprising lactose in a gas pressure measurement system.

97. The composition according to any one of paragraphs 78-96, wherein the composition is in a single dosage form.

98. The composition according to any one of paragraphs 78-97, wherein the single dosage form of the composition is capsules, powders, microcapsules, tablets, dragees, pellets and granules.

99. The composition according to any one of paragraphs 78-98, wherein the amount of the one or more Ligilactobacillus salivarius strains is in a range of 1E+08 Colony Forming Unit (CFU) to 1E+11 CFU.

100. The composition according to any one of paragraphs 78-99, wherein the amount of the one or more Ligilactobacillus salivarius strains is in a range of 1E+09 CFU to 1E+11 CFU, preferably in the range of 1E+09 CFU to 1E 10 CFU, more preferably about 1E+09 CFU.

101. The composition according to any one of paragraphs 78-100, wherein the amount of the one or more lactases is at least 4500 Food Chemicals Codex (FCC) units.

102. The composition according to any one of paragraphs 78-101, wherein the amount of the one or more lactases is 10000 FCC units.

103. The composition according to any one of paragraphs 78-102, wherein the one or more lactases belong to the GH35 Family or GH2 Family.

104. The composition according to any one of paragraphs 78-103, wherein the one or more lactases are acid lactase and/or neutral lactase.

105. The composition according to any one of paragraphs 78-104, wherein the one or more lactases are fungal lactases.

106. The composition according to any one of paragraphs 78-105, wherein the one or more lactases is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12,
b. a polypeptide derived from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12, by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).

107. The composition according to any one of paragraphs 78-106, wherein the lactase is a polypeptide comprising, consisting essentially of, or consisting of SEQ ID NO:1

108. The composition according to any one of paragraphs 78-106, wherein the lactase is a polypeptide comprising, consisting essentially of, or consisting of SEQ ID NO:2.

109. The composition according to any one of paragraphs 78-108, wherein the one or more lactases is for fast acting support and/or effect on lactose, and one or more Ligilactobacillus salivarius strains is for long lasting support and/or effect on lactose.

110. The composition according to any one of paragraphs 39-109, wherein the one or more lactases breaks down lactose that the subject consumes immediately after consumption of the composition and the one or more Ligilactobacillus salivarius strains breaks down lactose that the subject consumes for a time period of at least up to 4 hours after consumption of the composition, preferably lactose that the subject consumes for a time period of up to 24 hours after consumption of the composition.

111. The composition according to any one of paragraphs 39-110, wherein the one or more Ligilactobacillus salivarius strains breaks down lactose that the subject consumes for a time period of at least up to 8 hours after consumption of the composition.

112. The composition according to any one of paragraphs 39-111, wherein the one or more Ligilactobacillus salivarius strains breaks down lactose that the subject consumes for a time period of at least up to 12 hours after consumption of the composition.

113. A method for relieving from or preventing lactose induced abdominal discomfort; digesting lactose or improving digestion of lactose; providing digestive comfort after digestion of lactose on consumption of products containing lactose; and/or providing long-lasting support and/or effect for lactose digestion; the method comprising providing to a subject in need thereof an effective amount of one or more Ligilactobacillus salivarius strains or a composition comprising the one or more Ligilactobacillus salivarius strains.

114. The method according to paragraph 113, wherein the effect of the one or more Ligilactobacillus salivarius strains or the composition comprising such, after consumption, is for a time period of at least up to 4 hours, preferably for at least up to 8 hours.

115. The method according to any one of paragraphs 113 or 114, wherein the effect of the one or more Ligilactobacillus salivarius strains or the composition comprising such, after consumption, is for a time period of up to 24 hours.

116. The method according to any one of paragraphs 113-115, wherein lactose induced abdominal discomfort is diarrhea, constipation, bloating and/or gas induced from ingestion of products comprising lactose.

117. The method according to any one of paragraphs 113-116, wherein the Ligilactobacillus salivarius strains or composition are provided from 1 to 3 times daily to a subject, preferably at least once daily to a subject.

118. The method according to any one of paragraphs 113-117, wherein the Ligilactobacillus salivarius strain expresses one or more lactases.

119. The method according to any one of paragraphs 113-118, wherein the amount of lactases expressed from the Ligilactobacillus salivarius strain is measured in vitro based on the amount of lactose reduced from a lactose medium on which the Ligilactobacillus salivarius strain is grown.

120. The method according to any one of paragraphs 113-119, wherein the amount of lactose reduced from the lactose medium is at least 30%, preferably 75%.

121. The method according to any one of paragraphs 113-120, wherein the amount of lactose reduced from the lactose medium is at least 50% in a time period of up to 24 hours, preferably 75%.

122. The method according to any one of paragraphs 113-121, wherein the Ligilactobacillus salivarius strain has a deposit accession number DSM 34078, or the Ligilactobacillus salivarius strain is Ligilactobacillus salivarius strain having all the identifying characteristics of Ligilactobacillus salivarius strain having a deposit accession number DSM 34078, or a mutant thereof.

123. The method according to any one of paragraphs 113-122, wherein the Ligilactobacillus salivarius strains have the maximum growth rate of at least 5.0 mean optical density/minute in a lactose medium.

124. The method according to any one of paragraphs 113-123, wherein the Ligilactobacillus salivarius strains have a delta OD600 value of at least 0.7 in a lactose medium.

125. The method according to any one of paragraphs 113-124, wherein the Ligilactobacillus salivarius strains have the maximum growth rate of at least 5.0 mean optical density/minute in a lactose medium and have a delta OD600 value of at least 0.7 in a lactose medium.

126. The method according to any one of paragraphs 113-125, wherein the Ligilactobacillus salivarius strains survive in the gastrointestinal tract.

127. The method according to any one of paragraphs 113-126, wherein the Ligilactobacillus salivarius strains survive in the gastrointestinal tract for at least 4 hours.

128. The method according to any one of paragraphs 113-127, wherein the Ligilactobacillus salivarius strains grow on lactose in vitro and/or in the gastrointestinal tract of a subject.

129. The method according to any one of paragraphs 113-128, wherein the Ligilactobacillus salivarius strains prefer to grow on lactose in the presence of other sugars.

130. The method according to any one of paragraphs 113-129, wherein the Ligilactobacillus salivarius strains reduce gas production of fecal matter comprising lactose in a gas pressure measurement system.

131. The method according to any one of paragraphs 113-130, wherein the amount of the one or more Ligilactobacillus salivarius strains is in a range of 1E+08 Colony Forming Unit (CFU) to 1E+11 CFU.

132. The method according to any one of paragraphs 113-131, wherein the amount of the one or more Ligilactobacillus salivarius strains is in a range of 1E+09 CFU to 1E+11 CFU, preferably in the range of 1E+09 CFU to 1E 10 CFU, more preferably about 1E+09 CFU.

133. The method according to any one of paragraphs 113-132, wherein the composition further comprises one or more lactases.

134. The method according to any one of paragraphs 133, wherein the one or more lactases belong to the GH35 Family or GH2 Family.

135. The method according to any one of paragraphs 133 or 134, wherein the one or more lactases are acid lactase, and/or neutral lactase.

136. The method according to any one of paragraphs 133-135, wherein the one or more lactases are fungal lactases.

137. The method according to any one of paragraphs 133-136, wherein the one or more lactases is a polypeptide selected from the group consisting of:
a. a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12,
b. a polypeptide derived from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 or SEQ ID NO:12, by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
c. a polypeptide derived from the polypeptide of (a) or (b), wherein the N- and/or C-terminal end has been extended by addition of one or more amino acids, and
d. a fragment of the polypeptide of (a), (b), or (c).

138. The method according to any one of paragraphs 133-137, wherein the lactase is a polypeptide comprising, consisting essentially of, or consisting of SEQ ID NO:1.

139. The method according to any one of paragraphs 133-138, wherein the lactase is a polypeptide comprising, consisting essentially of, or consisting of SEQ ID NO:2.

140. The method according to any one of paragraphs 133-139, wherein the amount of the one or more lactases is at least 4500 Food Chemicals Codex (FCC) units.

141. The method according to any one of paragraphs 133-140, wherein the amount of the one or more lactases is 10000 FCC units.

142. The method according to any one of paragraphs 133-141, wherein the one or more Ligilactobacillus salivarius strains or the composition is provided 1 to 3 times daily to a subject, preferably daily at least once to the subject.

143. The method according to any one of paragraphs 133-142, wherein the one or more Ligilactobacillus salivarius strains or the composition is provided orally to the subject.

144. The method according to any one of paragraphs 133-143, wherein the one or more lactases breaks down lactose that the subject consumes immediately after consumption of the composition and the one or more Ligilactobacillus salivarius strains breaks down lactose that the subject consumes for a time period of at least up to 4 hours after consumption of the composition, preferably that the subject consumes for a time period of up to 24 hours after consumption of the composition.

145. The method according to any one of paragraphs 133-144, wherein the one or more Ligilactobacillus salivarius strains breaks down lactose that the subject consumes for a time period of at least up to 8 hours after consumption of the composition.

146. The method according to any one of paragraphs 133-145, wherein the one or more Ligilactobacillus salivarius strains breaks down lactose that the subject consumes for a time period of at least up to 12 hours after consumption of the composition.

### EXAMPLES

The following examples are not intended to be a detailed catalogue of all the different ways in which the present disclosure may be implemented or of all the features that may be added to the present disclosure. Subjects skilled in the art will appreciate that numerous variations and additions to the various embodiments may be made without departing from the present disclosure. Hence, the following descriptions are intended to illustrate some particular embodiments of the invention and not to exhaustively specify all permutations, combinations and variations thereof.

Unless otherwise indicated, the percentages set forth in the following examples are by weight, based upon the total weight of the composition.

### Material and Methods

Standard media were used for growing Ligilactobacillus strains, for example a medium providing 15-30 g peptone or tryptone per litre and 5-30 g glucose per litre. For preparing the standard media and the standard procedure was applied (concentration of components, sterilization, inoculation) and the microorganism was allowed to grow at elevated temperature (e.g., 35-42 °C) for sufficient time (e.g., 8-48 hours) unless otherwise stated.

### Example 1—Screening for Suitable Ligilactobacillus Strains

Ligilactobacillus strains were screened for lactose metabolization capacity using the screening protocol outlined below. 850 Ligilactobacillus strains were screened to select the strains that were able to grow on lactose as single C-source at very high rate and high optical density by analyzing the optical density at 595 nm (OD595) or 600 nm (OD600) as an indicator.

### Screening Protocol for Ligilactobacillus Strains Growing on Lactose as a Single C Source (96-Well Plate):

### Day 1: Set Up Pre-Culture

Cryostocks of each Ligilactobacillus strain in De Man, Rogosa and Sharpe (MRS) medium containing 18% glycerol stored in 96-well plates were thawed and 10 microlitre (μl) were transferred to 200 μl of culture medium YDA medium [Yeast Extract—Dextrose-Acetate; the ingredients are the same as in YLA medium (defined above), with one modification where lactose is replaced by D-Glucose] in a flat-bottomed 96-well plate. The plate was sealed with an airtight film and incubated for 24 hours at 37 °C under anaerobic conditions.

### Day 2: Set Up Main Culture

Plate 1 with Lactose:

5 μl of the 1:40 diluted pre-culture was transferred to 200 μl of lactose medium (YLA) in a 96-well flat bottom plate. The plate was sealed with an airtight film and placed in a plate photometer such as BioTek EON Microplate UV/VIS reader at 37 °C for 24 hours.

Optical density at OD600 nm was measured every 5 minutes. The growth rate in mean optical density per second (mOD/sec) was calculated using the linear region of the growth curve using the microplate reader software.

Plate 2 without Lactose:

5 μl of the 1:40 diluted pre-culture was transferred to 200 μl of the same medium without lactose. The plate was incubated under the same conditions as plate 1.

### Result

Although several species and plenty of strains display high cell density at the end of the cultivation time (delta OD600>0.7), only few are characterized by a high growth rate (Max growth rate<5) at the same time. Thus, in order to analyse and select the strains displaying superior lactose metabolization capacity, the strains with superior cell density were plotted on the X axis (delta OD600>0.7) and strains that displayed max growth rate (Max growth rate<5) were plotted on the Y axis enabling us to better analyse and select strain with the best lactose metabolization capacity.

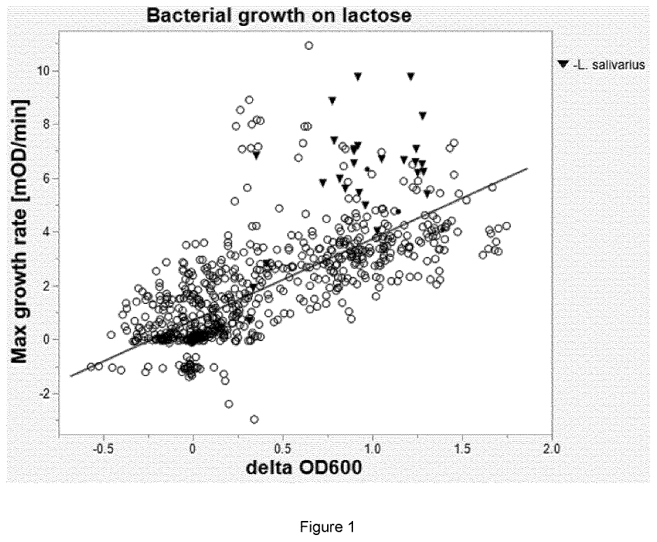

Out of 850 tested strains of various species, a total of 48 strains displayed high lactose metabolization capacity based on superior cell density (delta OD600>0.7) and max growth rate (max growth rate<5) as represented in FIG. 1.

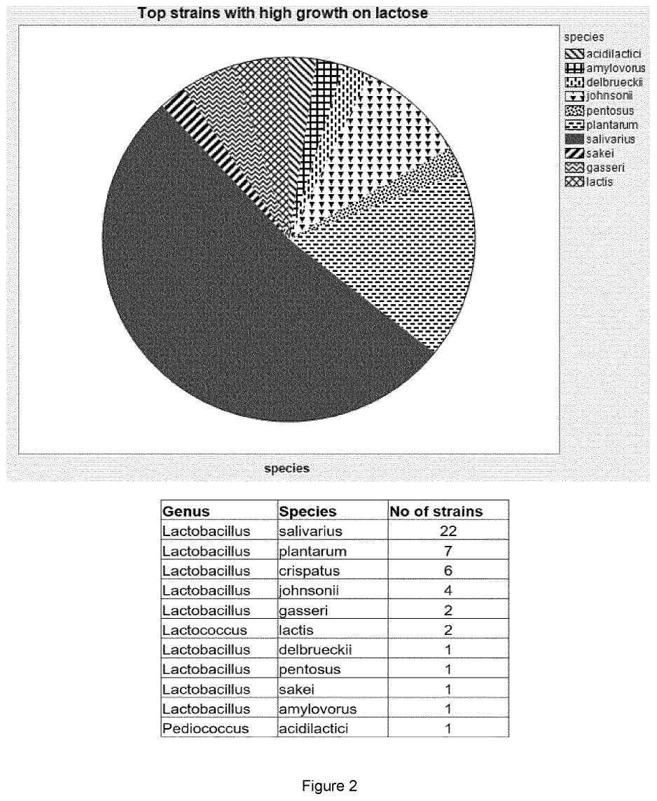

The species displaying lactose metabolizing characters are shown in FIG. 2 (pie chart and table).

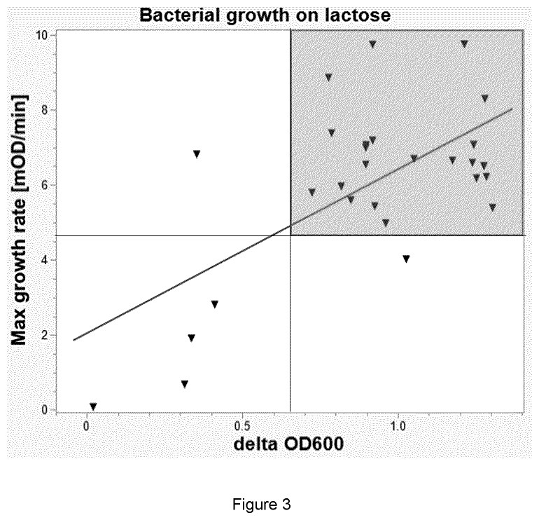

A majority of the tested L. salivarius strains (25-26 out of 28) grew at medium to high or very high growth rate and to high optical densities when lactose was the main carbon source as demonstrated in FIG. 3 (solid triangles).

Interestingly, in a total of 28 L. salivarius tested in the strain panel, 22 L. salivarius strains demonstrated superior lactose metabolization, i.e., superior cell density (delta OD600>0.7) and max growth rate (max growth rate<5) as represented in FIG. 3. These 22 L. salivarius strains represent ~46% of the top lactose metabolizers identified out of all the 850 screened Ligilactobacillus strains.

### Example 2—Growth Kinetics

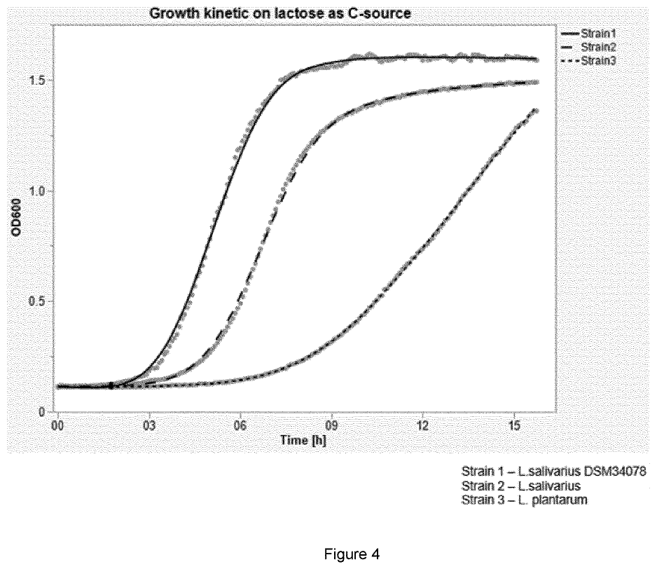

Single wells of a 96 deep well plate was inoculated with target bacterial strains for 24 hours in anaerobic conditions. On the next day, 5 μl of the culture was transferred into 200 μl fresh YLA medium in a 96 well plate. The plate was sealed with an air-tight foil and the plate was placed in a plate photometer for 17-24 hours. The optical density at 600 nanometer (nm) was continuously monitored and recorded to generate growth curves. The increasing optical density correlates with the number of bacterial cells in the solution over time. The maximal growth rate is determined by calculating the slope of an asymptote in the linear range of the growth curve. Strain 1 and Strain 2 are L. salivarius strains and Strain 3 is L. plantarum strain in FIG. 4. Strain 1 is L. salivarius DSM 34078. FIG. 4 demonstrates the growth kinetics of L. salivarius strain DSM 34078 on lactose as carbon source (C source) compared to some other candidates. Strains 1 and 2 shows positive density with little lag time (maximum density) and steep curve (maximum growth rate) demonstrating better growth kinetics than Strain 3. L. salivarius DSM 34078 (strain 1) has higher growth rate compared to the other L. salivarius strain (strain 2) and the L. plantarum strain (strain 3).

### Example 3—Gas Pressure Tests of L. salivarius DSM 34078

The L. salivarius strain deposited as DSM 34078 was tested in a pressure measurement assay with fecal samples. The pressure was continuously measured within the observation period using inhouse developed pressure measuring devices (2ML-FPM). Human fecal samples were collected using Gut Alive Kits. Samples were processed within two days after collecting by diluting fecal samples with glycerol/water in the ratio of 1:4 resulting in 25% fecal samples stored at −80 °C after preparation until using. The fecal slurry from human donors was resuspended in phosphate-buffered saline (PBS) and 5 gram per litre (g/l) lactose was added. All procedure steps were performed in an anaerobic atmosphere. The tubes were closed airtight and a sensor to continuously measure the pressure in headspace was inserted through the rubber septum. The pressure was measured for 24 hours and compared to pressure buildup by the microbial community without lactose addition. Next to lactose, 108 colony forming units (CFU) of selected bacterial strains shown to have high capacity to metabolize lactose was added to the microbiota and inoculated for 24 hours in anaerobic conditions with continuous pressure measurement.

TABLE 2

| | Fecal | | | |
|---|---|---|---|---|
| | sample | Lactose | L salivarius | |
| | 2.5% | 5 g/L | 1E+8 CFU/mL | |
| Approach | Final | Final | Final | PBS |
| Donor (D) | Yes | No | No | Yes |
| Donor + Lactose (D + S) | Yes | Yes | No | Yes |
| Donor + Lactose + | Yes | Yes | Yes | Yes |
| L. Salivarius DSM | | | | |
| 34078 (D + S + P) | | | | |

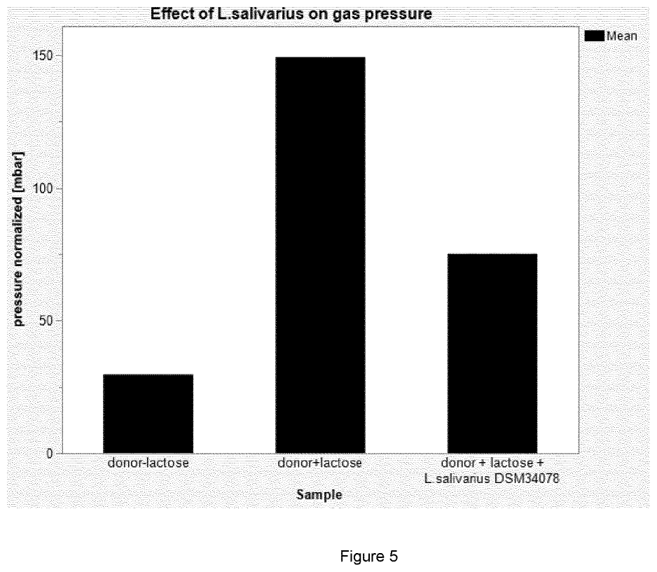

FIG. 5 shows that addition of L. salivarius DSM 34078 reduces lactose induced gas pressure in a fecal samples. FIG. 5 shows the effect of fermentation of human fecal slurry alone (donor), effect of addition of lactose to the fecal slurry on gas pressure creation (donor+lactose) and then further addition of L salivarius DSM 34078 on lactose induced increase in gas pressure (donor+lactose+L. salivarius DSM 34078). The addition of L. salivarius DSM 34078 significantly diminished gas pressure development by the microbiota by 50.36%. The pressure was calculated and represented in a bar chart as cumulated area under the curve (cAUC) in the presence of a human fecal donor set after 10 hours of incubation. Each column represents the mean of four fecal donors each tested twice.

### Example 4—Gas Pressure Tests of Different L. salivarius Strains

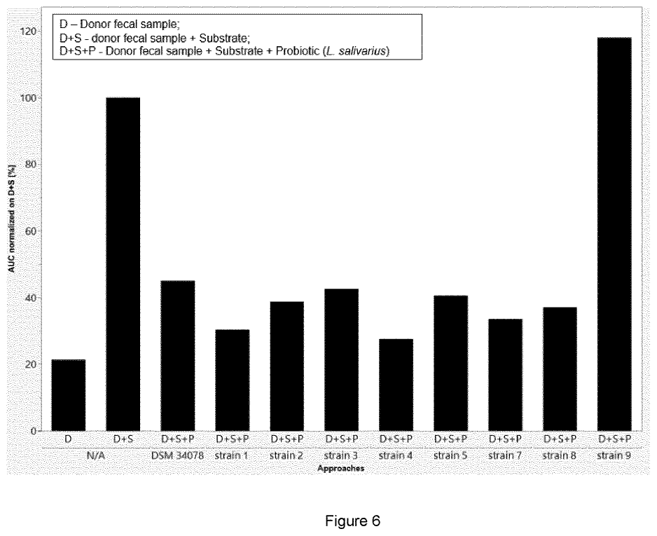

The gas pressure test performed in Example 3 was repeated with sample of Donor 9 with different strains of L. salivarius. Results presented in FIG. 6 show that 8 of 9 L. salivarius strains were able to reduce the gas pressure by 50-70%. All strains tested in gas pressure showed high growth rate in growth assay on lactose as a single carbon source, demonstrating high lactose metabolization capacity as shown in Table 3.

In this gas pressure assay, the fecal microbial community create a highly competitive environment and the hypothesis for the outlier could be that this strain might not be able to compete sufficiently with the fecal microbial community of Donor 9. Strain number 6 is L. salivarius DSM 34078.

TABLE 3

| Strain number | Mean growth rate [mOD/min] |
|---|---|
| 1 | 6.70 |
| 2 | 8.69 |
| 3 | 7.19 |
| 4 | 5.86 |
| 5 | 8.33 |
| 6 | 7.58 |
| 7 | 7.27 |
| 8 | 8.60 |
| 9 | 7.77 |

### Example 5—Identification and Quantification of DSM 34078 in Fecal Fermentation by Real-Time qPCR

To demonstrate that L. salivarius DSM34078 is able to proliferate in a fecal microbial community, the experimental setup described in Example 3 was analyzed by TaqMan qPCR using the strain specific primer and probe.

500 μl of fecal fermentations of each of four donors were sampled at time point 0 hours and at 24 hours. The bacterial cells were pelleted by centrifugation. The DNA was extracted by using a DNeasy® 96 Blood & Tissue Kit (Qiagen) according to the manufacturer's guidelines for gram-positive bacteria.

The qPCR was performed on qTower Real-Time-PCR cycler (Analytic Jena) using innuMIX qPCR 2× Master Mix Probe (Analytik Jena). Each reaction contained 2 μl of DNA template, 0.30 μM of each primer and 0.20 μM probe. The PCR cycling conditions included initial denaturation at 95 °C for 3 min followed by 40 cycles of denaturation at 95 °C for 10 s, annealing and extention at 60 °C for 45 s.

The standard curve was performed using serial dilutions of DNA extracted from 1E+09 cells of DSM 34078. Threshold cycle (Ct) value were ploted against log 10 of known cell number of bacteria present in PCR reaction.

Results were analyzed by linear regression to calculate the slope. The PCR amplification efficiency (E) was determined by equation E=10−1/(−slope)−1.

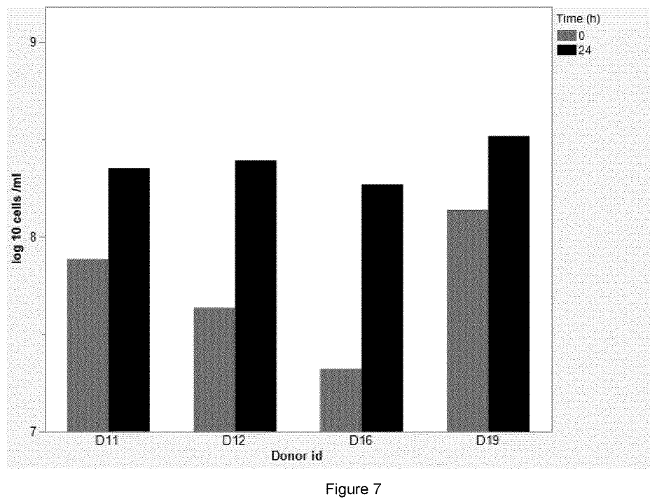

FIG. 7 shows that at time point 0 hours, the L. salivarius DSM 34078 was added to each donor fermentation in similar amounts of approximately 7.5-8 log cells/ml. After 24 h of fermentation with lactose, an increase of up to 1 log was observed in each donor tested. The L. salivarius DSM 34078 not only survived but also proliferated in the fecal microbial community.

DNA Sequence of Ligilactobacillus salivarius: SEQ ID NO: 13

Forward primer:
(SEQ ID NO: 14)
GGTTTCCCGGCCATTGAAAGG
Reverse primer:
(SEQ ID NO: 15)
TCTTATCCAATGCACTGTCCGC
TagMan probe:
(SEQ ID NO: 16)
CGACGCATCAGCTGGCCGCA

### Example 6—Microbiome Analysis

The experimental setup described in Example 5 was sampled for metagenomic sequencing. The whole ribosomal RNA operon (rn) from the DNA sampled in Example 5 was amplified using unique barcode primers for each sample. Each PCR reaction mix consisted of 300 nM each of the forward and reverse primer, 30 ng of template DNA and 20 μl of NEBNext Ultra II Q5 mix (New England Biolabs). The PCR cycling conditions were an initial denaturation at 98 °C for 30 s, followed by 25 cycles of 98 °C for 10 s, 59 °C for 20 s, and 72 °C for 2 min 30 s and final extension at 72 °C for 4 min.

The PCR products were purified with MagSi-NGS magnetic beads and quantified with Qubit Fluorometric Quantification HS (Invitrogen, California, USA). The library was prepared by mixing equimolar amounts of each purified amplicon and then subjected to sequencing adapter ligation using the SGK-LSK-110 ligation sequencing kit (Oxford Nanopore Technologies) and the NEBNext ligation kit (New England Biolabs).

Sequencing was launched on a MinION Mk1c sequencer with a R9.4.1 flowcell. Basecalling was performed with guppy v6.0.1 basecaller (Oxford Nanopore Technologies) to transform raw fast5 sequencing files to fastq files. Demultiplexing was performed using guppy barcoder v6.0.1 (Oxford Nanopore Technologies). Reads were subsequently classified using the taxonomic classification system Kraken2 v.2.0.9 beta against the Refseq database. Samples with less than 2000 reads and rare species (<0.1% in relative abundance) were excluded from the analysis. Data were analyzed using Qiime2 plugins.

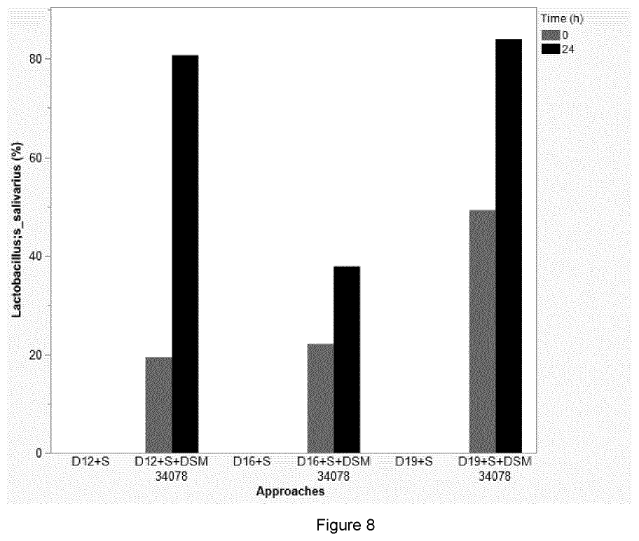

The relative abundance of L. salivarius species (Y-axis) was measured for each of three donors at time point 0 h (grey bars) and time point 24 h (black bars) and shown in FIG. 8. The different approaches are displayed on the X-axis: Donor+Lactose (denoted DX+S) and Donor+Lactose+L. salivarius DSM 34078 (denoted DX+S+DSM 34078) at time point 0 hours and at time point 24 hours. As expected, the probiotic strain was not detected in any of the Donor+Lactose approaches. In the approaches Donor+Lactose+L. salivarius DSM 34078, a sharp increase in relative abundance of probiotic strains was observed after 24 hours of fermentation compared to time point 0 hours.

### Example 7—Repeated Lactose Challenge

In the gastro-intestinal tract probiotics are exposed to conditions that may limit their survival and activity, such as chemical composition of the intestinal microenvironment, substrate fluctuation and constant wash out due to the transition of the intestinal content from the stomach to the colon. These parameters can only partially be implemented into a batch cultivation system. To demonstrate that L. salivarius DSM 34078 is able to persist in the small intestine, metabolize lactose and respond to repetitive lactose challenge, as it will in vivo, the behaviour of L. salivarius strain was explored in intestinal fluids, under continuous flow and upon repeated lactose challenge.

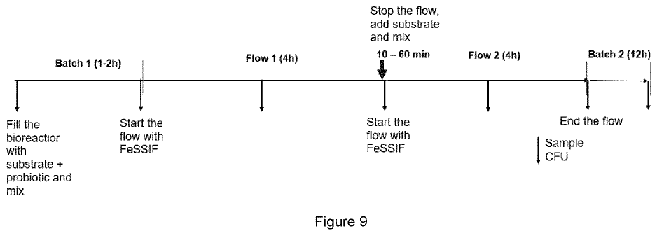

For this purpose, a bioreactor was filled with FeSSIF (Fed State Simulated Intestinal Fluid, commercially available from Biorelevant), milk (final lactose concentration of 10 g/L), yeast extract and PBS-resuspended freeze-dried powder of L. salivarius (total cell count 10\hat{ }7-10\hat{ }9). Upon mixing and heating (37 °C) the bacterial cells remain in the batch culture for 1 or 2 h. After this time the flow was switched on and FeSSIF without substrate was used as feed. The flow rate was chosen to ensure a complete exchange of the bioreactor volume within 4 h time to simulate the transit time in the small intestine. After 4 h the flow was stopped and the substrate in form of skim milk powder and yeast extract diluted in PBS was added to the fermenter to achieve a final concentration of 10 g/l lactose. After an hour of rest, the flow was switched on for additional 4 h. Samples were taken every 1 to 2 hours to determine cell number of L. salivarius during the experiment. FIG. 9 represents the experimental design in a flow diagram.

In some experiments, L. salivarius cell count dropped within the first 1-2 h of the experiment. A possible explanation can be lack of sufficient metabolic activity for fast adaptation of the bacterial cells to FeSSIFE, due to a short rehydration time of the freeze-dried powder before addition to the bioreactor.

During the first period of continuous flow cell number of L. salivarius did not decrease or even increased up to 0.5 log, depending on the bacterial count at the beginning. This demonstrates that despite a continuous wash out of the biomass and growth substrate, L. salivarius proliferates so rapidly in FeSSIF that it can persist in the bioreactor.

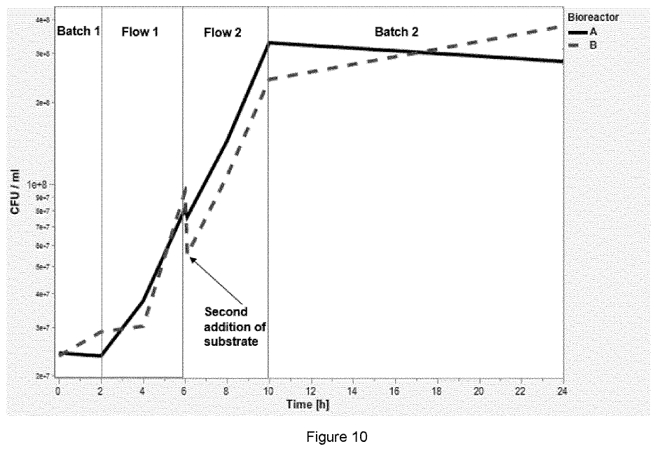

After the second addition of the substrate, growth of L. salivarius was observed at least with equal or even higher growth rate, so that in all experiments determined cfu at the end of the continuous flow (4 h after repeated substrate addition) was at least 0.4 log higher. This demonstrates that the strain is resistant to substrate fluctuation. Based on the increasing proliferation rate in the course of the experiment, it is concluded that the strain adapts to the intestinal conditions simulated by FeSSIF. In FIG. 10, the graph represents CFU time dependency of two bioreactors A and B under the same conditions (technical replicates).

Thus, this indicates that the strain may efficiently colonize the human small intestine, as it can adapt to the chemical composition of intestinal fluid within few hours even when the substrate concentration is periodically very low. There was retention of the strain in the bioreactor despite the absence of any surfaces suitable for bacterial attachment. The condition in the bioreactor differs from the small intestinal environment where mucus is available as possible attachment site for the strain. Additionally, in the chosen experimental setup the focus was on mimicking the small intestinal transition time of 4 h rather than aligning the flow rate to the real conditions. For this reason, the applied flow rate was probably higher than the physiological flow rate.

In the next set of experiments, there is a plan to extend the adaptation time of L. salivarius and modifying the flow rate to mimic the physiological values with the goal of demonstration of lactose metabolization upon repeated lactose challenge upon continuous flow.

### DEPOSIT OF BIOLOGICAL MATERIAL

The following biological material has been deposited under the terms of the Budapest Treaty with Deutsche Sammlung von Mikroorganismen und Zellkulturen GmbH (DSM), Mascheroder Weg 1 B, D-38124 Braunschweig, Germany, and given the following accession number:
Deposit: Lactobacillus salivarius
Accession Number DSM 34078
Date of Deposit 21 Oct. 2021

The source and geographical origin of Ligilactobacillus salivarius with accession Number DSM 34078 is chicken feces and Germany respectively. The strain has been deposited under conditions that assure that access to the culture will be available during the pendency of this patent application to one determined by foreign patent laws to be entitled thereto. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

1-10. (canceled)

11: A method for relieving from or preventing lactose induced abdominal discomfort; said method comprising:
providing to a subject in need thereof an effective amount of one or more Ligilactobacillus salivarius strains or a composition comprising the one or more Ligilactobacillus salivarius strains.

12: The method of claim 11 wherein the Ligilactobacillus salivarius strain has a deposit accession number DSM 34078, or the Ligilactobacillus salivarius strain is Ligilactobacillus salivarius strain having all the identifying characteristics of Ligilactobacillus salivarius strain having a deposit accession number DSM 34078, or a mutant thereof.

13: The method of claim 11, wherein the composition further comprises one or more lactases.

14: The method of claim 11, wherein the one or more lactases is a polypeptide selected from the group consisting of:
a. a polypeptide having at least 80% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12;
b. a polypeptide derived from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12;
c. a polypeptide derived from the polypeptide of (a) or (b), wherein the N- and/or C-terminal end has been extended by addition of one or more amino acids; and
d. a fragment of the polypeptide of (a), (b), or (c).

15: A Ligilactobacillus salivarius strain having a deposit number DSM 34078 or a Ligilactobacillus salivarius strain having all the identifying characteristics of Ligilactobacillus salivarius strain having a deposit accession number DSM 34078, or mutant thereof.

16: A composition comprising one or more Ligilactobacillus salivarius strains of claim 15; and one or more lactases.

17: The composition of claim 16 wherein the one or more lactases is a polypeptide selected from the group consisting of:
a. a polypeptide having at least 80% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12;
b. a polypeptide derived from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12 by having 1-30,
c. a polypeptide derived from the polypeptide of (a) or (b), wherein the N- and/or C-terminal end has been extended by addition of one or more amino acids, and
d. a fragment of the polypeptide of (a), (b), or (c).

18: The composition according to claim 16, wherein the amount of the one or more Ligilactobacillus salivarius strains is in a range of 1E+08 Colony Forming Unit (CFU) to 1E+11 CFU.

19: The composition according to claim 16, wherein the amount of the one or more lactases is at least 4500 Food Chemicals Codex (FCC) units.


SEQUENCE LISTING (16 sequences)

SEQ ID NO: 1 - protein, 987 aa, Aspergillus candidus
ASIKHRLNGFTILEHPDPAKRDLLQDIVTWDDKSLFINGERIMLFSGEVHPFRLPVPSLWLDIFHKIRALGFNCVSFYIDWALLEGKPGDYRAEGIFALEPFFDAAKEAGIYLIARPGSYINAEVSGGGFPGWLQRVNGTLRSSDEPFLKATDNYIANAAAAVAKAQITNGGPVILYQPENEYSGGCCGVKYPDADYMQYVMDQARKADIVVPFISNDASPSGHNAPGSGTGAVDIYGHDSYPLGFDCANPSVWPEGKLPDNFRTLHLEQSPSTPYSLLEFQAGAFDPWGGPGFEKCYALVNHEFSRVFYRNDLSFGVSTFNLYMTFGGTNWGNLGHPGGYTSYDYGSPITETRNVTREKYSDIKLLANFVKASPSYLTATPRNLTTGVYTDTSDLAVTPLMGDSPGSFFVVRHTDYSSQESTSYKLKLPTSAGNLTIPQLEGTLSLNGRDSKIHVVDYNVSGTNIIYSTAEVFTWKKFDGNKVLVLYGGPKEHHELAIASKSNVTIIEGSDSGIVSTRKGSSVIIGWDVSSTRRIVQVGDLRVFLLDRNSAYNYWVPELPTEGTSPGFSTSKTTASSIIVKAGYLLRGAHLDGADLHLTADFNATTPIEVIGAPTGAKNLFVNGEKASHTVDKNGIWSSEVKYAAPEIKLPGLKDLDWKYLDTLPEIKSSYDDSAWVSADLPKTKNTHRPLDTPTSLYSSDYGFHTGYLIYRGHFVANGKESEFFIRTQGGSAFGSSVWLNETYLGSWTGADYAMDGNSTYKLSQLESGKNYVITVVIDNLGLDENWTVGEETMKNPRGILSYKLSGQDASAITWKLTGNLGGEDYQDKVRGPLNEGGLYAERQGFHQPQPPSESWESGSPLEGLSKPGIGFYTAQFDLDLPKGWDVPLYFNFGNNTQAARAQLYVNGYQYGKFTGNVGPQTSFPVPEGILNYRGTNYVALSLWALESDGAKLGSFELSYTTPVLTGYGDVESPEQPKYEQRKGAY

SEQ ID NO: 2 - protein, 1304 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSERVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 3 - protein, 988 aa, Penicillium cremeogriseum
AENQTTTEWPLHDDGLNQVVQWDHYSFQINSQRIFIFSGEFHYWRIPVPGLWRDILEKIKAVGFTAFAFYSSWAYHAPNNQTLDFTTGAHDFTPLFELAKELGLYIIVRPGPYVNAEASAGGFPLWLTTGDYGTLRNNDTRYTKAWTPYFTKMSQITSKYQVTDGQNAIVYQIENEYGEQWVGSASKRVPNEKAINYMELLEANARANGITVPLTANDPNMNSHSWGSDWSKEGGNVDVAGVDSYPSCWTCDLSQCTSTNGEYIPFQVMDYYDYFQESQPTMPEFMPEFQGGSYNPWGGPEGGCAENSNQDFANLFYRWNIGQRVTAMSLYMLFGGTNWGAIAAPVTGSSYDYSAPISEDRSIGDKYYETKLLALFTRCAKDLTMTNLIGNGTQYTDNGNVRAYELRNPDTNAGFYATFHTNTSVSTNEAFHLKVNTSAGALTVPTHGGVVRLNGHQSKILVTDFTFGKETLLYSTAEVLTYAVFDKTPTLVLWMPTGETGEFNIKGAKKGSVQKCQGCSSVKFFKEHGGLTAAITQSSGMSVLAIDDIQVIVLDRTSAYKFWAPALTNDPLVPETESVLVQGPYLVRGASLSGSKLAVTGDIINATTLEVFAPKAVKSITWNGKTLKTQRTEYGSLKGSIAAPKAVTLPSFKSWKSKDSLPERLADYDDAGAAWVDANHQSTLNPRAPTTLPVMYADEYGFHNGVRLWRGYFNGSATGAYINVQGGYAFGWSAWLNGQFIGSYLGSADLESGNLSLSFTNATINTNKPNILLIVHDDTGHDETSGALNPRGILDAHLLGSSSGFTHWRLAGTAGGESNLDPVRGVFNEDGLYGERVGWHLPGYDDSTWSSSGGSSLSFTGATVRFFRTTIPLNFPSNTDVSISFILSTPSGSTTAYRAQIFVNGYQYGRYNPYIGNQVVYPVPAGILNYSGENTVAVAVWAQTEEGARMEVDWRVNYVADSSLDVVSVSKKAEGLRTKWTEVREKFA

SEQ ID NO: 4 - protein, 1007 aa, Aspergillus carneus
AQNHTDSEWPLHDNGLNTVVQWDHYSFQVHGQRIFVFSGEFHYWRIPVPGLWRDILEKIKAAGFTAFAFYSSWAYHAPNNHTVDFSTGARDITPIYELAKELGMYIIVRPGPYVNAEASAGGYPLWVTTGAYGSLRNDDARYTEAWKPYFAKMSEITSQYQVTDGHNTFCYQIENEYGQQWIGDPVDRNPNQTAVAYMELLEESARENGIVVPLTANDPNMNTKSWGNDWSNAGGNVDVVGLDSYPSCWTCDVTQCTSTNGEYVPYKVMGYYDYFQEVQPTMPGFMPEFQGGSYNPWAGPEGGCPGDTGVDFANLFYRWNIAQRVTAMSLYMLYGGTNWGAIAAPVTATSYDYSSPISEDRSIGSKYYETKLLALFTRSATDLTMTDRIGNGTQYTNNPAVAAYELRNPVTNGAFYVTIHADSTVGSDESFRLNVNTSAGAFTVPSKGAIRLNGHQSKIVVTDFRFGPSHSLLYSTAEVLTHAVFDKQATIVLWVPTGESGEFAVKGAKSGKVESCPRCSNATFTRKKDVLVVNFTQTGGMSVLQLNNGVRVVLLDRPAAYNFWAPPVTDDPFAPETDLVLVQGPYLVRSASLSGSTLALRGDSIKETALEIFAPKKVKTVTWNGKRVKTSKSSYGSLTASLAPPPAVTLPALTSTRWKSQDSLPERLPSYDDSGPAWVDADHMTTQNPRTPETLPVLYADEYGFHNGIRLWRGSFTDAASGVYLNVQGGAAFGWSAYLNGHFLGSHLGTATTSQANKTLVFPTGALNQNATNTLLVIHDDTGHDQTTGALNPRGILAARLLPASNAAADSTAPTFTRWRVAGTAGGESDLDPVRGVYNEDGLFAERVGWHLPGFDDGAWPAANTTAVAQETGTVSLSFTGATVRFFRTVIPLHLPRGIDASISFVLGTPAGTSTAYRAQLFVNGYQYGRYYPHIGNQVVYPVPAGVLDYDGENTIGVAVWAQSDAGAAINLDWRVNYVADSSLDAVRLTGEGSLRPQWSEARERYA

SEQ ID NO: 5 - protein, 991 aa, Aspergillus wentii
ASIKHRINGFTLTEHPDQAKRDLLQKHVTWDDKSLFVNGERIMIFSGEVHPFRLPVQSLWIDVFQKIKALGFNCVSFYTNWALLEGKPGEYRADGVFALEPFFDAAKEAGIYLLARPGPYINAEVSGGGFPGWLQRVNSTLRTSGKTYLESTDNYIAHIAATIAKAQITNGGPVILYQPENEYTGACCGYDDFPDGGYMQYVMDQARKAGIVVPMISNDASPKGTNVPGSGEGAVDIYGHDSYPLGFDCANPTVWPDGSLPTNFRDLHLQQSPSTPYSLIEFQAGAYDPWGGPGFDACAGLVNHEFERVFYKNDFSFGVAILNLYMTFGGTNWGNLGHPGGYTSYDYGSPLTETRNITREKYSELKLIGNFAKVSPSYLLATPGNLTTSAYTNNAELAVTPLVGHGSGSYFVVRHSDYTSQASTDYKLRLPTSAGNLTVPQLGGTLSLNGRDSKIHVVDYNVSGTNVLYSTAEVFTWKKFADKKVLVLYGGPDEHHELAISSKSNVSVIEGSESGISSKQIKNTVVVGWDVSSTRRIIQVDDLQVLLLDRNSAYNYWVPQLPAEGTSPGYSTQKNTASSIIVNAGYLVRTAYVDGQDLHLTADFNATTTVEVIGAPSSVKNFYINEKQIQHSVDKNGIWSGKVEYTAPKIELPSLKDLDWKYLDTLPEIQSSYDDSAWTAADLKKTDNTLRPLDTPTSLYSSDYGFHAGYLIYRGHFTATGNESSLFIHTQGGSAFGSSVWLNETYVGSWAGIDKDMDSNSTYKLPKLQGGKPYVFTVVVDNMGLDEDWTVGSDEMKDPRGILGYKLSGRDSSAITWKLTGNLGGEDYQDKTRGPLNEGGLFSERQGFHQPQPPSDDWESSSPFDGLTKPGIGFYSAKFDLDIPTGWDVPLFFNFANSTSPPPAYRVQLYVNGYQYGKYVNNVGPQQSFPVPEGILNYRGTNWLAVTLWALESDGAKLDGLELANTTPVLTALKDLEASEQPKYEKREGAY

SEQ ID NO: 6 - protein, 994 aa, Rasamsonia byssochlamydoides
HVERQRGAESQWPLHDDGLNKVVQWDHYSFLLNGERIFLFSGEFHYWRIPVPELWIDILQKIKAAGFTAFTFYLNWAYHAPNNETLDFSTGVHDFTPLFKIAKELGLYVFVRPGPYINAETSAGGFPLWMTTGAYGTLRNDDTRYTAAWTPYFSNVSQITSQYQITKGQNVIAYQIENEYGEQWIGNPSQRVPSETAVHYMELLEANARANGINVPLTANEPNMYSISWGKDWSNAGGNVDITGLDSYPSCWTCDLSQCTSVNGEYIPFQVVDYYDYFQQTQPTMPSFMPEFQGGSYNPWGGPEGGCPDNTGPDFANVFYRWNIGQRVTAMSLYMLYGGTNWGAIATPVTATSYDYSAPISEDRSIGAKYYETKLLALFTRTARDLTVTDLIGNGTQYITNAAVKAYEIRNSNTNAGFYVTIHTNSSLATTETFQLHVQTSAGNLTVPRHGGIITLDGHQSKIIVTDFTFGNETLLYSTAEVLTYAVLDGVPTLALWVPTGESGEFAVKGAKYGSVKRCQGCSGVGFYPENGGVTVTFTQAEGMSVVELDSGSRILLLDRTFAYLFWAPSLSNDPSAPENESILVQGPYLVRTAAIAGSTISLTGDSTNASTIVEVFAPKNVDTVSWNGQTLKTSKTAYGSLQATLPGLPPVKLPVLNTWKWNDSLPERFPNYDDSGAAWVDANHMTTPNPSPPATLPVLYADEYGFHNGVRLWRGYFNGTATGVFLNVQGGYAFGWSAWLNGEFLGSYLGDASLEQGNLTLSFNNVTVNDSSPNVLLVVHDDTGHDETTGALNPRGILQATLLSPDNTTTTQFTHWRVAGTAGGESNLDPVRSSYNEDGLYAERVGWHLPGFDDSAWAATTSFPSLSFSGAGVRFYRTVVPLNIPAGVDVSISFVLSAFGSTKALRAQLFVNGYQYGRFNPYIGNQVVFPVPPGILDYAGENTIGLAVWAQTEAGASVSVDWQVNYVAESSLDVTFDGSALRPGWSAERLKYA

SEQ ID NO: 7 - protein, 994 aa, Aspergillus cervinus
AQNQSDTQWPLHNDGLNKVVEWDHYSFLVNDQRIFIFSGEFHYWRIPVPELWRDILEKIKAAGFTAFAFYSNWAYHAPNNHTVDFSTGAHDITPIFELAKELGMYIIVRPGPYINAEASAGGFPLWLTTGAYGSLRNDDPRYTAAWTPYFSNMTQITSKYQLTNGQNTFCYQIENEYGYQWIGDPSARDPNTTGIAYMELLEAAARENGITVPLTANDPNMGTFAWGSDWSNAGGNVDVTGVDSYPSCWTCDVSQCTSTNGEYVAYEVEDYYDYFQEFQPTMPSFMPEFQGGSYNPWAGPEGGCPEDTGADFANLFYRWNIGQRVTAMSLYMLYGGTNWGALAAPVTATSYDYSSPISEDRSIGSKYYETKLLSLFTRTAKDLTMTDLIGNGTQYTTNAAVRAFELRNPDTNAGFYATFHLDTTIGTNESFKLHVNTSIGALTVPQYGGSIQLNGFQSKILVTDFTFGSNTLLYSTAEVLTYATLDNSQTIVLWVPTGESGEFAVKGAKLGSVQNRQGSSSIKFYQEKGYLVVGFTQASGMSVLQLDNGVRVILLDRTAAYNFWVPALTDDPFAPENEIVLVQGPYLVRSSSLSKSTLALRGDSINATTLEVFASEAVNAITWNGKPLKTSRTLYGSLKASIAAPLPIQLPALTSWKSSDSLPEKLATYDDSGAAWVDANHMTTLNPSPPATLPVLYADEYGFHNGIRLWRGYFNGTASSVFLNIQGGTAFGWSAWLNGQFLGSYLGDASIEQAGQTLSIPSSALSTSSPNVLLVVHDDTGHDETTGALNPRGILEAQLISADNATQFTHWRVAGTAGGESNLDPIRGAWNEDGLYAERMGWHLPGFDDSAWTGTSLSSSSSSLSFTGATVRFFRTVVPLQIPEGVDVSISFVLSTPTANNATNAYRAQLFVNGYQYGRYNPYIGNQVVFPVPPGILNYQGENTIGLAVWAQSEAGASIGLDWQVNYVAESSLSVASFGAGLRPAWTEERLQYA

SEQ ID NO: 8 - protein, 998 aa, Aspergillus oryzae
QQNSTRWPLHDNGLTDTVEWDHYSFLINGQRHFVFSGEFHYWRIPVPELWRDLLEKIKAAGFTAFSIYNHWGYHSPKPGVLDFENGAHNFTSIMTLAKEIGLYMIIRPGPYVNAEANAGGLPLWTTTGAYGKLRDNDPRYLEALTPYWANISKIIAPHLITNDGNVILYQIENEYAEQWLDEETHEPNTSGQEYMQYLEDVARENGIDAPLIHNLPNMNGHSWSKDLSNATGNVDVIGVDSYPTCWTCNVSECASTNGEYIPYKTLIYYDYFKELSPTQPSFMPEFQGGSYNPWGGPQGGCPDDLGPDFANLFYRNLISQRVSAISLYMLYGGTNWGWHASTDVATSYDYSSPISENRKLIEKYYETKVLTQFTKIAQDLSKVDRLGNSTKYSSNPAVSVAELRNPDTGAAFYVTQHEYTPSGTVEKFTVKVNTSEGALTIPQYGSQITLNGHQSKIIVTDFKFGSKTLLYSTAEVLTYAVIDGKEVLALWVPTGESGEFTVKGVNSAKFADKGRTANIEIHPGTNNVTVSFMQRSGMSLVELGDGTRIVLLDRSAAHVFWSTPLNNDPAEAGNNTVLVHGPYLVRSAKLEGCDLKLTGDIQNSTEVSIFAPKSVCSVNWNGKKTSVKSAKGGVITTTLGGDAKFELPTISGWKSADSLPEIAKDYSATSKAWVVATKTNSSNPTPPAPNNPVLYVDENDIHVGNHIYRATFPSTDEPPTDVYLNITGGRAFGYSVWLNSDFIGSWLGTATTEQNDQTFSFSNATLSTDEDNILVVVMDNSAHDLRDGALNPRGITNATLIGPGSYSFTEWKLAGNAGFEDHLDPVRAPLNEGSLYAERVGIHLPGYEFDEAEEVSSNSTSLTVPGAGIRVFRTVVPLSVPQGLDVSISFRLTAPSNVTFTSAEGYTNQLRALLFVNGYQYGRFNPYIGHQIDFPVPPGVLDYNGDNTIAVTVWSQSVDGAEIKVDWNVDYVHETSFDMNFDGAYLRPGWIEERREYA

SEQ ID NO: 9 - protein, 1008 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYVPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 10 - protein, 887 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

SEQ ID NO: 11 - protein, 1025 aa, Kluyveromyces lactis
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

SEQ ID NO: 12 - protein, 1710 aa, Niallia circulans
MSKTTSAAGNSVSYDGERRVNFNENWRFQRETNGSIAGAQNPGFDDSSWRKLNLPHDWSIELDFNKNSLATHEGGYLDGGIGWYRKTFTIPESMKGKRISLDFDGVYMNSTTYLNGEVLGTYPFGYNAFSYDISDKLYKDGRANVLVVKVNNTQPSSRWYSGSGIYRNVYLTVTDPIHVARYGTFVTTPNLEKSIKEDRADVNIKTKISNDAAEAKQVKIKSTIYDGAGNTVQTVETEEKTAAAGTVTPFEQNTVIKQPKLWSIDKPYRYNLVTEVIVGGQTVDTYETKFGVRYFKFDENEGFSLNGEYMKLHGVSMHHDLGALGAATNARGVERQMQIMKDMGVNAIRVTHNPASPELLEAANKLGLFIIEEAFDSWAQSKKPYDYGRFFNAWAEHDIKEMVDRGKNEPAIIMWSIGNEIYDTTNAAGVETARNLVGWVKEIDTTRPTTIGEDKTRGDKVNVTPINSYIKEIFNIVDVVGLNYSENNYDGYHKQNPSWKLYGSETSSATRSRGVYTHPYQYNQSTKYADLQQSSYDNDYVGWGRTAEDAWKYDRDLKHIAGQFIWTGFDYIGEPTPYYNSYPAKSSYFGAVDTAGFPKDIFYYYQSQWKKEPMVHLLPHWNWKEGEKVRVLAYTNASKVELVLNGESLGEKNYDNKQTSWGAPYKETKDGKTYLEWAVPFKPGKLEAVAKDENGKVIARDQVVTAGEPASVRLTADRKVVKADGTDLSFITADIVDSKGIVVPDADHLITFNVTGQGELAGVDNGNASSVERYKDNKRKAFSGKALAIVQSSKLSGKITVHASVAGLSSDSTSVFTVTPADHDKKIVAGIDDVNLTVDVNEAPKLPSEIKVYYSDESAAAKNVTWDEVDPKQYSTVGEFTVEGSVEGTSLKAKAFVIVKGIVAVKPYSTATKVGVQPVLPEKATLLYSDGTTKGATVTWDEIPEDKLAKEGRFTVEGSVEGTDLKANVYVRVTNEVKSVNIMLQEQGSAYPKLEATFTNPADNLQHLNDGIKSYTNNPVNRWTNWTRTPRDAGDSITVNFGKKHVINNLDLFVFTDSGTVVPEKAEVQYWDGTAWKDVENLTQPSPYVVEKNELTFDAVATEKLKFHLTPSVKGKFLALTEAEVYADQIVMGETAKLQSITVNGKALEGFDHAKKNYELVLPYGSELPKIEAAAADNATVTILPAFSYPGTAKLFVTSEDGKVTTEYSIGVSTEEPKLVSAELSADKTNVMEDDIIDLKVIGLFESKEKIDVTDSQPTYEFDQQIIKIEGNKLYALETGNVKVKVTVTYKGVSVTTPALEFTIAKNPAPKYITSLEPVTVVVKKGEAPELPATVVAHYNRGIPRDVKVKWERINPSKYQQLGEFTVSGMVEGTDIKAQAKVIVKGAVAVEDIRMAVLLKQMPQLPGKVTVYYSDGAEEQRAVKWEEIPQEELENVGEFKVKGDVNGVKLKATATIRVTDEVGGEQNISRAKNGYEYPKAEASFTNNGPGSSDRIEAINDDVISYEANPHNRWTNWQPVPRAGDWVSITFGDYEPTEYDVDSMEIHWFADHGTSYPERFQIEYKSGDSWKEVTSLKSDPASPALGKANVYSFDRVKTSAIRVKMTAQAGKSLAITELKVFSKWPKAGTEPEVTDIKVGGKSILEDFEQKGDHYEVTIDAGDANVMPKINVKAKDQTSITIVPAVTSPSTAKVIAKSEDGKKVKVYSIHYK

SEQ ID NO: 13 - DNA, 1476 bp, Ligilactobacillus salivarius
  source 1..1476: mol_type = genomic DNA
gagaactagactaagccactttggcttttggtctagttctcttttttgataattcacgaatcatgagactagaagtcttcaacgaaaccagaattctaatcctaaagtggaagaagttacggaagccatacgcagtccgcttgattactttgattttattattggtgccttcgatcggcccattggttagtcgggtgtaaaaactggcaataatctcctggcagtgcttgacgagtgttcgcttaactcgtcttatagcgtttggtaaattattccgcttgattaactgatctaacaaagcctgactatggtgactaattgcggttattaagtcttgatagtattcgtaggcagtccttagctctgaagagaagttaagcagccgttcgacgacttcttgattggacaaacgagcatgctcaaagttacggcgtgaatagtagcgcagataatctaactgcccttccttagccattaataacttccaaaaatgctttaaagcacggtattcatgcgtatttgccccgtattctttcatgaccttaagccggacactttgaagtgcccggtaggcctgaacgacgacgtggaaatgatcagctacgataatcgctttgggaaataattcatgaatgaggttacgataaggttcaaacagatcgactacgacgatcttaactgaccagcgggccttcttagaatagtgggaaagaaagtagctgcgcataaagcgtgaattacgggactgaatgatatcaatagtgcgatggttctgtggattcattaaaatcatactcatcttactttgggcgaacttgccggatttaaagtcatcaaaagcgatggttgccggtagccagtgaggattagttttaaaggtatcctcgagtccctcgagctgacgctcaacggtgttaatagaaacattgtgatggctggcaatagtccgcattgattcattctcgctgagttcctgcacaatatggtatttaacgatattcgcaattcgacaggcaaatttaactccttggacttcggcaaccttggtaacgcgcttgggacattgagctgatggtggacagagatattgttgtttcctgattttcaagacagctggtttcccggccattgaaaggatcttgatcgttactggcttcttacggaaaccgttacgacgcatcagctggccgcaaagcggacagtgcattggataagattgaatcaaatgaatgatctttagttctttatcctcagttaaaaatggtttgtcaggttttaagtgaggatccttgattccagtccaaaagttgatatcattagtcatgaggaatatatcctttctaagacaactgagtggttatggggataacttaagacagttgtctttttcatttttaaattaatggtatagcaagtaacaaaaaacggtaaagataagaaatcatcttatccttaccgaatatcatagagccaaat

SEQ ID NO: 14 - DNA, 21 bp, Ligilactobacillus salivarius
  source 1..21: mol_type = other DNA
ggtttcccggccattgaaagg

SEQ ID NO: 15 - DNA, 22 bp, Ligilactobacillus salivarius
  source 1..22: mol_type = other DNA
tcttatccaatgcactgtccgc

SEQ ID NO: 16 - DNA, 20 bp, Ligilactobacillus salivarius
  source 1..20: mol_type = other DNA
cgacgcatcagctggccgca